(19) United States
(12) Patent Application Publication
(10) Pub. No.: US 2025/0132049 A1
(43) Pub. Date: Apr. 24, 2025

(54) PERFORMING PREDICTIVE HEALTH RISK MODELING AND RISK MITIGATION TRADE-OFF ANALYSIS

(71) Applicant: Aon Consulting, Inc., Chicago, IL (US)

(72) Inventors: Todor Penev, Atlanta, GA (US); Ronald J. Ozminkowski, Saline, MI (US); Meghan Rausch, Kansas City, MO (US); Anthony Persico, Denver, CO (US); Leanne Metcalfe, Cedar Creek, TX (US); Jonathan Bowen, Denver, CO (US); Jax Li, Atlanta, GA (US); Jae Hyung Lee, Atlanta, GA (US); Mingmin Chiang, Singapore (SG); Dipti R. Patel, Chicago, IL (US)

(73) Assignee: Aon Consulting, Inc., Chicago, IL (US)

(21) Appl. No.: 18/908,302

(22) Filed: Oct. 7, 2024

Related U.S. Application Data
(60) Provisional application No. 63/591,381, filed on Oct. 18, 2023.

(51) Int. Cl. G16H 50/30; G16H 40/20; G16H 50/70

(52) U.S. Cl. CPC G16H 50/30; G16H 40/20; G16H 50/70

(57) ABSTRACT

In an illustrative embodiment, systems and methods for deriving risk patterns corresponding to a set of medical conditions through analyzing historic treatment of a population of individuals involve segmenting treatment data records of the population into a set of risk segments, analyzing the treatment data records to identify risk patterns indicative of each of a set of medical conditions, and calculating relative impact of each of the set of medical conditions based at least in part on prevalence of each medical condition among the population.

### RELATED APPLICATIONS

This application claims the benefit of and priority to U.S. Provisional Patent Application Ser. No. 63/591,381, entitled “Performing Predictive Health Risk Modeling and Risk Mitigation Trade-Off Analysis” and filed Oct. 18, 2023. All above identified applications are hereby incorporated by reference in their entireties.

### BACKGROUND

Future health outcomes for individuals are difficult to predict. Health outcomes requiring greater resource allocations, such as serious diseases, acute incidents (e.g., injuries, etc.), and chronic disorders, can lead to volatility within an organization's infrastructure. For example, budgeting and/or staffing may be affected. Space for treating and/or housing additional patients may be needed. The inventors recognized a need for greater insight into future resource allocations attributed to predicted disease and/or disorder within a population. The inventors further recognized a need to identify potential mitigation efforts in light of predicted health concerns.

### SUMMARY OF ILLUSTRATIVE EMBODIMENTS

In one aspect, the present disclosure relates to systems and methods for deriving risk patterns corresponding to a set of medical conditions through analyzing historic treatment of a population of individuals. The system may include data storage for storing treatment data records collected over at least a threshold period of time and demographic data records including demographic information for each member of a population represented in the treatment data records. The system may include risk forecast models trained, using historic claims data corresponding to a large population of individuals, to predict a likelihood of each of a set of medical conditions based on treatment data analysis. For example, each risk forecast model may be configured to identify at least one risk pattern corresponding to at least one medical condition. The system may be configured to perform operations (e.g., of the method), via hardware and/or software logic, including segmenting the treatment data records into sets of segmented treatment data records according at least to a set of demographic groupings, and applying one or more sets of the segmented claims data to each risk forecast model to identify a portion of members as a respective clinical population being likely to have or develop the respective each of the set of medical conditions. The operations may include analyzing information corresponding to each respective clinical population to determine one or more resource forecasts for treating the respective clinical population over at least one predetermined future time period. The forecast, for example, may include an estimated cost, an estimated staffing need, an estimated equipment need, and/or an estimated clinical resource need. The operations may include prioritizing, based on the one or more resource forecasts, at least one of set of medical conditions or the portion of the members. In this manner, various embodiments disclosed herein provide the advantage of objectively analyzing historic data to forecast a future need that can be used, in some examples, in budgeting, planning, preparing, purchasing, and/or hiring decisions to prepare for providing for the upcoming medical needs of a population.

In some embodiments, the set of medical conditions includes at least one of one or more diseases or one or more chronic disorders. At least a portion of the set of demographic groupings may each correspond to at least one risk forecast model of the set of risk forecast models. The treatment data records may include medical insurance claims records.

In some embodiments, the operations include segmenting the treatment data records according to a set of medical condition groupings, where the medical condition groupings each correspond to one of a set of preexisting medical conditions. The set of medical conditions may include at least a portion of the preexisting medical conditions. The operations may include analyzing the treatment data records to identify, for one or more of the members, information identifying at least one of diagnosis or treatment of at least one medical condition of the set of medical conditions. The demographic data records may include, for each member of a portion of the members, identification of one or more preexisting conditions of the set of preexisting medical conditions.

In some embodiments, prioritizing the at least one of the set of medical conditions or the members includes identifying a top segment of the set of medical conditions according to greatest cost. By focusing on the medical conditions associated with the greatest cost, for example, budgeting and purchase planning may be focused in the area that will best cover the future needs of the organization (e.g., medical facility, etc.).

In one aspect, the present disclosure relates to systems and methods for deriving risk patterns corresponding to a set of medical conditions through analyzing historic treatment of a population of individuals. The system may include data storage configured to store medical claims data records collected over at least a threshold period of time, the medical claims data records relating to members of a population. The system may include hardware logic and/or software logic configured to perform operations (e.g., the method) including categorizing the medical claims data records according to one or more characteristics, the characteristics including at least one of a corresponding encounter type of a set of encounter types, a corresponding diagnostic type of a set of diagnostic types, a corresponding therapy type of a set of therapy types, or a corresponding medication type of a set of medication types. The operations may include identifying, within the medical claims data records, one or more codings indicative of each of a set of medical conditions. The operations may include identifying risk patterns by analyzing, for each respective medical condition of the set of medical conditions, a portion of the medical claims data records corresponding to each member of the population having codings indicative of the respective medical condition to recognize one or more risk patterns. For each risk pattern, the operations may include calculating one or more impact values, each impact value corresponding to a resource allocation or a forecast outcome. The operations may include arranging the risk patterns by impact according to at least one of the one or more impact values. The risk patterns, in some examples, may focus the end user (e.g., medical service provider, employer, governmental agency, insurance group, etc.) on prime areas to focus on for mitigating future risk. Interventional programs, for example, may reduce the propensity of one or more behaviors identified within a risk pattern. In another example, promoting a treatment pattern identified as resulting in lower risk to the patient may result in lower loss of life and/or greater quality of life for future patients.

In some embodiments, the operations further include, after categorizing the medical claims data records, filtering the plurality of medical claims data records to remove one or more encounter types and/or one or more diagnostic types representative of pre-diagnosis events. The set of medical conditions may include one or more of at least one disease, at least one chronic condition, at least one acute condition, or at least one disorder. By remove the diagnostic or pre-diagnostic encounter types, for example, the data may be targeted to analyze only those treatment patterns related to a patient that has already been diagnosed with a particular disease, condition, or disorder.

In some embodiments, the operations include dimensionally reducing the risk patterns into a set of grouped risk patterns, each group risk pattern representing a respective two or more risk patterns having a common medical condition type. The common medical condition type may be one of a type of disease, a type of disorder, a type of treatment, or a type of therapy. The common medical condition type may be one or more of a cancer type, a cardiovascular type, a neurological type, an upper gastro-intestinal/esophageal type, a diabetes type, a musculoskeletal type, a hypertension type, a metabolic disorder type, a mental health disorder type, a lipid disorder type, a neurodivergent diagnoses type, or a pulmonary type. Dimensionally reducing the risk patterns may include aggregating a respective impact value of each risk pattern of the two or more risk patterns to calculate a group impact value. In grouping risk patterns, for example, very similar medical codings (e.g., duplicative or near-duplicative across various medical groups or insurance providers) may be combined to obtain a truer picture of the impact and treatment of the disease, condition, or disorder as a whole. Similarly treated diseases, disorders, or conditions may be analyzed, in another example, as a category to identify synergies in risk reduction across a broader category of patients.

In some embodiments, the data storage is further configured to store demographic data records including demographic information for each member of a population, where each record correlates to a corresponding portion of the medical claims data records. Identifying the risk patterns may include analyzing, for each respective medical condition of the set of medical conditions, the portion of the medical claims data records in view of demographic information derived from at least a portion of each of the demographic data records corresponding to the portion of the medical claims data records. The data records may be de-identified to protect the identities of underlying patients while providing rich truth data to the methods and systems described herein.

The foregoing general description of the illustrative implementations and the following detailed description thereof are merely exemplary aspects of the teachings of this disclosure and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1 is a block diagram of an example platform and environment for performing health risk analysis on a population;

FIG. 2A and FIG. 2B illustrate a flow chart of an example method for extracting risk patterns from insurance claims data;

FIG. 2C illustrates a flow chart of an example method for applying dimensionality reduction to identify most impactful risk patterns;

FIG. 3 is an example graphical user interface screen for presenting predictive health risk analysis related to a subject population;

FIG. 4A and FIG. 4B are example graphical user interface screens for presenting overview predictive information related to multiple risk segments derived from a subject population through health risk analysis;

FIG. 5A through FIG. 5C illustrate example graphical user interface screens for presenting risk optimization trade-offs to alter outcomes predicted in the health risk analysis;

FIG. 6 is a screen shot of an example graphical user interface screen for risk financing and risk budgeting;

FIG. 7 is a flow diagram of an example process for performing health risk analysis and risk mitigation analysis for a subject population;

FIG. 8 is a flow diagram of an example process for generating risk forecast models for use in the process of FIG. 7; and

FIG. 9 is an example graphical presentation representing a scatter plot of members of a population according to risk representations.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

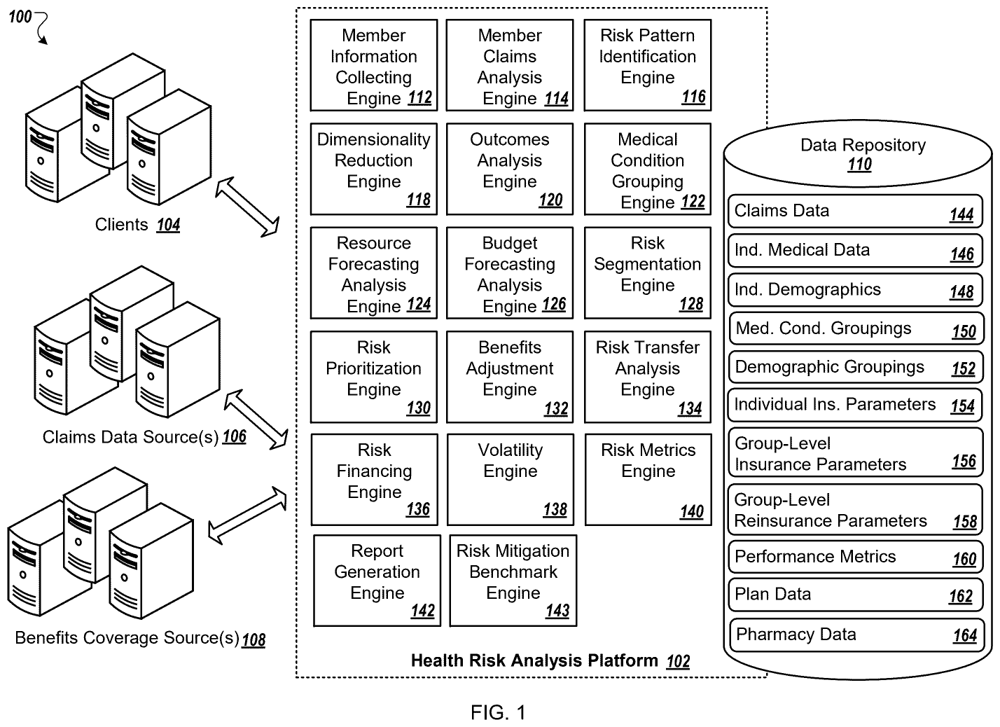

FIG. 1 is a block diagram of an example platform 102 and environment (e.g., system) 100 for performing health risk analysis on a population. Clients 104 may access the health risk analysis platform 102 to obtain an objective assessment of likely future risks (e.g., in a next month or number of months, in a next calendar year, in a next business year, over a next two calendar years, over a next two business years, etc.) in health outcomes and/or health care expenditures based on member population information. The clients 104, in some examples, may include employers, health plans, insurers, care management solutions, organizations (e.g., governmental, educational, etc.), live-in nursing care facilities, assisted living care facilities, post-surgical rehabilitation centers, and/or addiction rehabilitation centers. The member population information, for example, may include member demographics, behavioral risk factors, and/or chronic condition factors. A portion of the member population information, for example, may be derived through past insurance claims obtained from one or more claims data sources 106.

In some implementations, the health risk analysis platform 102 models potential risks based on historic information derived from insurance claims data (e.g., claims data records) obtained from the one or more claims data sources 106. The health risk analysis platform 102, further, may analyze risks on costs in view of insurance mitigation (e.g., benefits coverage information obtained from one or more benefits coverage sources 108).

In some implementations, a member information collecting engine 112 identifies, gathers, and organizes data for a member population corresponding to at least one of the clients 104 for use in risk modeling and risk analyses by the health risk analysis platform 102. The member information collecting engine 112, for example, may identify demographic information for each member of the population such as, in some examples, an age or age range, a gender, a position in a family structure, and/or a geographic location (e.g., city, county, zip code, etc.). The demographic information, for example, may be organized as individual demographics data 148, stored in a data repository 110. The member information collecting engine 112, in another example, may identify individual insurance parameters 154 for certain members of the member population such as, in some examples, insurance eligibility and/or dependent status, as well as identifying benefits coverage (e.g., provider, plan, coverage level, etc.). In a further example, the member information collecting engine 112 may identify individual medical data 146 for various members of the population such as, in some examples, conditions, diseases, prescriptions, behavioral factors, and/or socioeconomic information (e.g., social determinants of health).

In some implementations, a member claims analysis engine 114 uses the individual insurance parameters 154 and individual demographics 148 to gather data useful in monitoring health status and/or medical care costs longitudinally across multiple months and benefit periods for each member and/or unit of risk. A unit of risk may be considered as an individually represented significant health risk factor, such that each member of the population could be calculated as zero, one, or many units of risk (e.g., one individual member may be considered as both a cancer unit of risk and a diabetes unit of risk). The data, for example, may include certain codings and/or categories of claims data from the claims data source(s) 106.

In some implementations, data obtained by the member information collecting engine 112 and the member claims analysis engine 114 is used by a risk pattern identification engine 116 to identify patterns of risk from the historical member data indicative of costly and/or serious medical concerns. The risk pattern identification engine 116, for example, may analyze medical encounter types (e.g., inpatient, outpatient, professional, etc.), pharmacy data 164 (e.g., prescriptions), and/or other benefits program encounters (e.g., laboratory screenings, imaging screenings, rehabilitation/physical therapy, etc.) for each member to identify a set of medical conditions (e.g., acute medical conditions, chronic health conditions, diseases, other medical disorders, etc.). In illustration, the risk pattern identification engine 116 may identify presence and stage of diabetes as well as types and stages of heart disease. During analysis, in some embodiments, the risk pattern identification engine 116 filters out noise (e.g., pre-diagnosis encounters) to produce estimates of a timing of initial diagnosis of one or more medical conditions. The risk pattern identification engine 116 may further pair the initial diagnosis timing with a disease stage presence that could be indicative of criticality/risk to the member.

The risk pattern identification engine 116 may produce a large number of patterns, each directed to a particular health condition and/or disease state. The high dimensionality of risk patterns can make modeling more difficult, as very specific patterns may be less applicable to a broader group of subjects in predicting health outcomes and/or patient resource needs based on the historic data. In some implementations, the risk patterns obtained by the risk pattern identification engine 116 are provided to a dimensionality reduction engine 118 to iteratively analyze the risk patterns to reduce the number of risk patterns to a set of broader risk groupings and/or most impactful (e.g., resource-wise, highest likelihood of permanent disability/death without swift intervention) risks for use in risk modeling. For example, the dimensionality reduction engine 118 may combine more refined cancer indications to a coarser cancer category.

The dimensionality reduction engine 118, in some embodiments, automatically reduces medical factors, such as condition identifiers and/or prescription identifiers, to group same or similar elements to assist in matching. For example, a same prescription drug may be provided in multiple forms and/or doses as well as by multiple manufacturers, such that a single type of antihistamine, for example, may be offered as over a dozen options. Similarly, condition identifiers, such as International Statistical Classification of Diseases and Related Health Problems (ICD) codes, a global standard released by the World Health Organization, may include tens or even hundreds of codes relevant to a particular chronic medical condition, such as diabetes or multiple sclerosis. Beginning with thousands or tens of thousands of condition identifiers associated with the individuals of a member population, the dimensionality reduction engine 118 may reduce the corresponding conditions to a corresponding hundreds of conditions or, in other words, the initial conditions may be reduced by about a factor of 10 to about a factor of 100. In some examples, the dimensionality reduction engine 118 may apply statistical analysis and/or machine learning classifiers to reduce the initial pool of condition identifiers.

The dimensionality reduction engine 118, in some implementations, reduces conditions in part by filtering out at least a portion of codes related to diagnostic services, such as laboratory service codes. For example, many diagnostic services relate to determining whether or not a patient has a condition, while those patients with the condition will receive additional services that relate mostly or solely to those with the given condition. In another example, identifiers corresponding to various condition types (e.g., cancer, cardiovascular disease, etc.) may be organized into hierarchical groupings, where each layer of the hierarchy includes one or more condition sub-types. In illustration, mental illness may include a hierarchical layer including substance abuse, depression, anxiety, adjustment disorder, and attention-deficit/hyperactivity disorder (ADHD).

In some implementations, an outcomes analysis engine 120 determines, for each risk pattern of the set of risk patterns produced by the risk pattern identification engine 116, one or more performance metrics 160 representative of relative patient care. The outcomes analysis engine 120, in some embodiments, categorizes the performance metrics 160 as being relevant to cost, resource utilization, and/or quality of care. In some illustrative examples, the performance metrics 160 may include timing and/or number of inpatient admissions, readmissions, office visits, emergency room visits, and/or preventative visits related to the subject medical condition of each respective risk pattern.

In some implementations, a medical condition grouping engine 122 analyzes individual medical data 146 and/or claims data 144 for each member of a client population to match each member with one or more medical condition groupings of a set of medical condition groupings 150. The medical condition groupings 150, for example, may correspond to information relevant to the set of risk patterns determined by the dimensionality reduction engine 118. The medical condition grouping engine 122, for example, may combine members' diagnoses (e.g., as determined by the member claims analysis engine 114 and/or the individual medical data 146) into the broader risk categories generated by the dimensionality reduction engine 118.

In some implementations, a resource forecasting analysis engine 124 models predicted distributions of resource allocations (e.g., types and numbers of encounters, estimated invoicing amounts, etc.) for a predetermined projection period. The predicted distributions, for example, may follow historic patterns of claims data entries for individuals with similar risk patterns. The projection period, in some examples, may include an upcoming quarter, six months, year, or two years.

In some implementations, a budget forecasting analysis engine 126 matches required resources to the predicted distributions to calculate a forecast resource allocation for each member or member segment for the prediction period. The required resources, in some examples, can include costs, staff members, equipment, inventory, and/or supplies for meeting the anticipated needs of the member/each member of the member segment based on the forecast risk distributions among the member population. In some embodiments, the budget forecasting analysis engine 126 applies plan data 162 corresponding to one or more benefits plans covering the member population to the predicted distributions to calculate predicted costs to the client 104. The plan data 162, for example, may be obtained from the client 104 and/or one of the benefits coverage sources 108.

In some implementations, a risk segmentation engine 128 accesses individual member claims distribution for the projection period, along with the members' medical condition groupings 150, demographic groupings 152, and individual medical data 146, to develop custom member risk segmentations for a member population associated with one of the clients 104. The risk segmentation engine 128, for example, may cluster and/or group the members based on the set of risk patterns determined by the dimensionality reduction engine 118. For example, the risk segments may include health condition (e.g., disease and/or disorder, etc.) groupings such as cancer, cardiovascular, neurological, musculoskeletal, diabetes, hypertension, metabolic disorders, mental health disorders, lipid disorders, neurodivergent diagnoses, and/or pulmonary disorders. The risk segmentation engine 128, in some embodiments, further clusters and/or groups the members based on type and/or level of risk. In some examples, the members may be grouped into risk severity categories (e.g., low, medium, high, etc.), risk type categories (e.g., acute but non-recurring, recurring, etc.), and/or risk forecast categories (e.g., rising risks, emerging conditions, therapy risks, etc.). Further, the risk segmentation engine 128, in some embodiments, groups members according to both disease and/or disorder groupings as well as one or more demographic sub-groupings (e.g., by gender, age range, geographic location, etc.) within certain disease and/or disorder groupings. The particular risk segments, for example, may be customized to a particular client, a particular type of client, and/or a composition of the subject population. In some embodiments, segments applied by the risk segmentation engine 128 may be updated to add/remove risk segments and/or refine the definition of one or more risk segments.

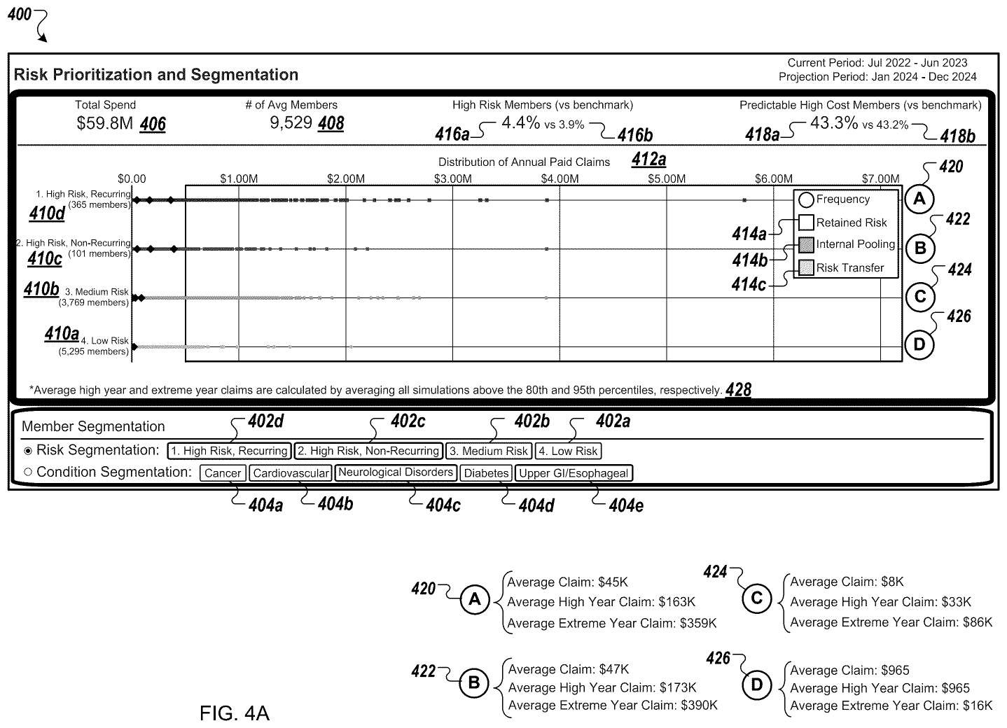

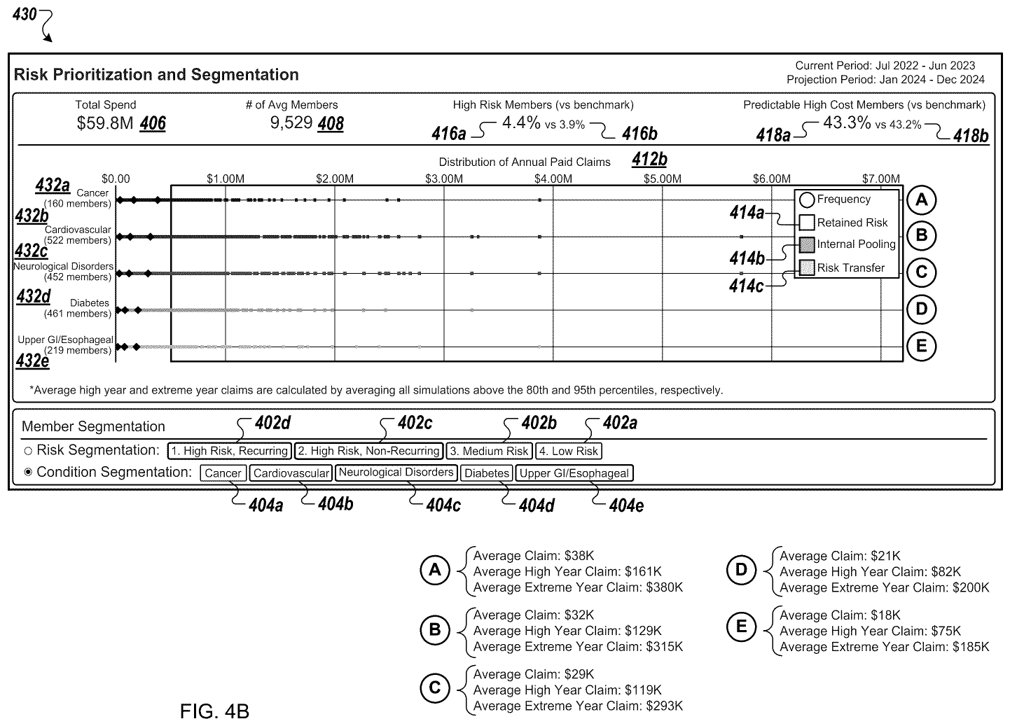

Turning to FIG. 4A, an example graphical user interface 400 presents a cost analysis related to predicted risk segments within a population 408 of 9,529 members. The GUI 400 presents two options for reviewing risk segment analysis, a risk level analysis 402 (selected) and a medical condition (e.g., disease and/or disorder) categorization analysis 404 (not selected). The risk level analysis 402 (e.g., “Risk Segment Present A”) includes low risk segment 402a, a medium risk segment 402b, a high risk, non-recurring segment 402c, and high risk, recurring segment 402d.

Turning to FIG. 4B, the medical condition categorization analysis 404 (e.g., “Risk Segment Preset B”) includes a cancer risk segment 404a, a cardiovascular risk segment 404b, a neurological disorder risk segment 404c, a diabetes risk segment 404d, and an upper GI/esophagus risk segment 404e. The condition segments 404, for example, may include the top five prioritized condition segments out of a set of many (e.g., a dozen, dozens, up to fifty, etc.) conditions and/or condition types analyzed by the system 100 of FIG. 1.

Returning to FIG. 1, in some implementations, risk segments of the member population generated by the risk segmentation engine 128 are provided to a risk prioritization engine 130 to identify, from the risk segments, a subset of highest priority risk segments. In some embodiments, the risk segmentation engine 128 identifies a subset of the members in need of prioritized care management. The risk prioritization engine 130, for example, may analyze the risk segments to identify those representing an addressable risk in which timely intervention is predicted to correspond to a substantial increase in likelihood of positive outcome and/or a substantial decrease in forecast resource allocations. In some embodiments, the risk segmentation engine 128 identifies a subset of highest cost risk segments. For example, the risk prioritization engine 130 may apply budget estimates generated by the budget forecasting analysis engine 126 to identify the highest cost members and/or member risk segments based on budget risk. The risk prioritization engine 130 may rank individual members and/or member risk segments from highest priority to lowest priority (e.g., in relation to anticipated aggregate cost, in relation to anticipated cost volatility, in relation to a number of members affected, and/or in relation to anticipated benefit in focusing in the near term on their care management, etc.). In some embodiments, the risk prioritization engine 130 identifies a subset of greatest staff and/or equipment/space needs. In some examples, to identify potential surges in hospital bed need, intensive care unit (ICU) equipment allocation, nursing hours, and/or surgical room staffing and/or equipping, the risk prioritization engine 130 may identify, on behalf of a clinical care type client (e.g., hospital, outpatient surgical facility, etc.), top ranking impacts to staffing, equipment, and/or room allocations. The risk prioritization engine 130 may be configured to quantify and rank a number of various resource needs (e.g., budget, equipment, staffing, real estate, consumables stocking, etc.). The resource needs, in some examples, may depend in part on a client type and/or composition of the subject population.

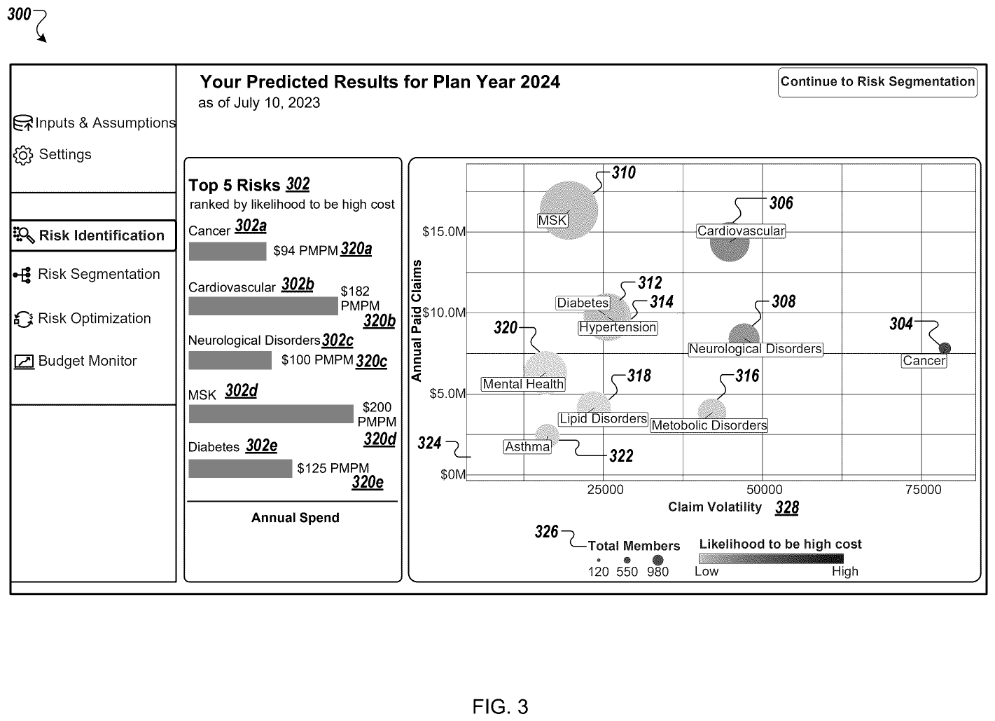

Turning to FIG. 3, an example graphical user interface (GUI) 300 for presenting risk identification information is illustrated. The GUI 300 presents a top five risks 302 to a member population, ranked according to likelihood of being high cost, including a cancer risk category 302a, a cardiovascular risk category 302b, a neurological disorders category 302c, a musculoskeletal category 302d, and a diabetes category 302e. The top five risks 302 may have been determined by the risk prioritization engine 130. Each of the top five risks 302 is presented alongside an estimated spend 320a-e represented as a per member per month (PMPM) value.

Returning to FIG. 1, in some implementations, a benefits adjustment engine 132 applies group-level insurance parameters 156 to model the effect of a large number (e.g., hundreds, thousands, etc.) of different benefits policies on modifying the client's budget, as forecast by the budget forecasting analysis engine 126. The benefits adjustment engine 132, for example, may allocate more costs to the members covered by the benefits plan(s) offered through the client 104 and/or allocate more costs to the client 104 to alleviate budget expenditures by the individual members. Further, the benefits adjustment engine 132 may identify plans providing better coverage to the member population based on the risk segments generated by the risk segmentation engine 128. In some examples, better coverage may be coverage more responsive to the anticipated needs of the member population, lower cost coverage, and/or coverage for particular interventions identified by the risk prioritization engine 130 as being important to improving the forecast health outcomes of the highest risk members of the member population. The benefits adjustment engine 132, in some embodiments, models perception trade-offs associated with various benefits options. Perception, for example, may represent anticipated relative approval of the membership population for each potential benefits option.

In some implementations, a risk metrics engine 140 generates a set of risk metrics associated with the analysis performed by the health risk analysis platform 102 regarding a particular member population. In some embodiments, the risk metrics engine 140 identifies a number of members estimated to be diagnosed with and/or treated for each health condition, health condition category, health disorder, and/or health disorder category represented in the risk groupings generated by the risk pattern identification engine 116 over a predetermined projection period. The risk metrics engine 140 may further calculate metrics to distinguish groupings of members according to risk, such as the frequency of simulated expenses exceeding a certain threshold, the portion of simulated expenses being recurring due to chronic disorder or condition, and/or average simulation costs per member. In illustration, each risk group may be defined based on the frequency at which a member's simulations exceeds a threshold value (e.g., in dollars). Further to the illustration, a low risk member's simulations may never exceed the threshold, while a medium risk member's simulations may exceed the threshold value less than a threshold percentage of time (occurrences), while a high risk member's simulations may exceed the threshold value at or above the threshold percentage of time. Further, certain chronic conditions may move a member to the high risk category regardless of simulation output due to the compounding cost year-over-year for the particular chronic condition. For example, the risk metrics engine 140 may calculate the total numbers of members per health condition category used to generate the relative sizes 326 of the plots of the graph of FIG. 3, the total number of members included in each risk category 410 (low risk category 410a having 5,295 members, medium risk category 410b having 3,769 members, high risk, non-recurring category 410c having 101 members, and high risk, recurring category 410d having 365 members) of FIG. 4A, and/or the total number of members included in each condition category 432 (e.g., cancer category 432a having 160 members, cardiovascular category 432b having 522 members, neurological disorders category 432c having 452 members, diabetes category 432d having 461 members, upper GI/esophageal category 432e having 219 members) of the user interface 430 of FIG. 4B. Further, the risk metrics engine 140 may calculate a percentage of high risk members 416 (4.4%) out of the total members 408, as illustrated in FIG. 4A and FIG. 4B. The risk metrics engine 140, in some embodiments, calculates an estimated cost per member per health condition category, such as the per member per month estimated costs 320a-e presented in FIG. 3. The risk metrics engine 140 may further calculate, from the estimated costs per member and/or condition category, an estimated budget such as a total spend 406 ($59.8 million) of FIG. 4A and FIG. 4B.

In some embodiments, the risk metrics engine 140 calculates typical or average claims values related to a member population, a benchmark population, and/or a subpopulation thereof (e.g., medical condition category, risk category, and/or demographic category, etc.). For example, turning to FIG. 4A, a set of high risk, recurring segment metrics 420 include an average claim cost of $45,000, an average high year claim cost of $163,000, and an average extreme year claim cost of $359,000. Further to the example, a set of high risk, non-recurring segment metrics 422 include an average claim cost of $47,000, an average high year claim cost of $173,000, and an average extreme year claim cost of $390,000. The example continues with a set of medium risk segment metrics 424 including an average claim cost of $8,000, an average high year claim cost of $33,000, and an average extreme year claim cost of $86,000. Finally, per this example, a set of low risk segment metrics 426 include an average claim cost of $965, an average high year claim cost of $965, and an average extreme year claim cost of $16,000. As stated in a note 428 presented in the user interface 400, “average high year and extreme year claims are calculated by averaging all simulations above the 80th and 95th percentiles, respectively.”

Returning to FIG. 1, a volatility engine 138, in some implementations, analyzes the claims data 144 (e.g., on a month-by-month basis, on a treatment path basis, etc.) to forecast annual seasonality and/or treatment trend patterns in various resource utilizations. The volatility engine 138, for example, may predict volatility in resource allocation (e.g., expenditures) per risk segment (e.g., medical condition or medical condition group). In illustrative examples, volatility can include volatility in relation to treatment location (e.g., inpatient vs. outpatient, health system v. independent facility, geographic disparities, etc.), seasonal volatility discovered in relation to certain diseases or disorders, and/or volatility in type of treatment pursued (e.g., pharmacological vs. surgical, etc.). Turning to FIG. 3, for example, a graph of claims per risk category (e.g., including the top risks 302a-e) presents claim volatility 328 (x-axis) in view of annual paid claims 324 (y-axis). The claim volatility 328 ranges from 0 to $75,000. Volatility, for example, may be calculated as the standard deviation of predicted payments for each condition and/or condition category. As illustrated, the claim volatility 328 is highest for cancer 304 and lowest for both mental health disorders 320 and asthma 322.

In some implementations, a risk transfer analysis engine 134 obtains forecast resource allocations for the member population from the budget forecasting analysis engine 126 and models a large number (e.g., hundreds, thousands, etc.) of different risk transfer policies for mitigating budget risk to the client 104. The risk transfer analysis engine 134, for example, may apply group-level reinsurance parameters 158 to model different risk attachment points, different cost coverage percentages, and/or different multi-segment coverage scenarios. The risk transfer analysis engine 134, in some embodiments, identifies a risk/reward trade-off at each level. For example, based on estimated total costs in view of risk transfer mitigation levels, the risk transfer analysis engine 134 may calculate a percentage of a client's budget left uninsured after transferring risk via each of the modeled risk transfer policies. The risk transfer analysis engine 134, in some embodiments, considers volatility in resource allocation, as generated by the volatility engine 138, to produce estimates based on a variety of scenarios (e.g., a typical year, a costly year, an extreme year, etc.).

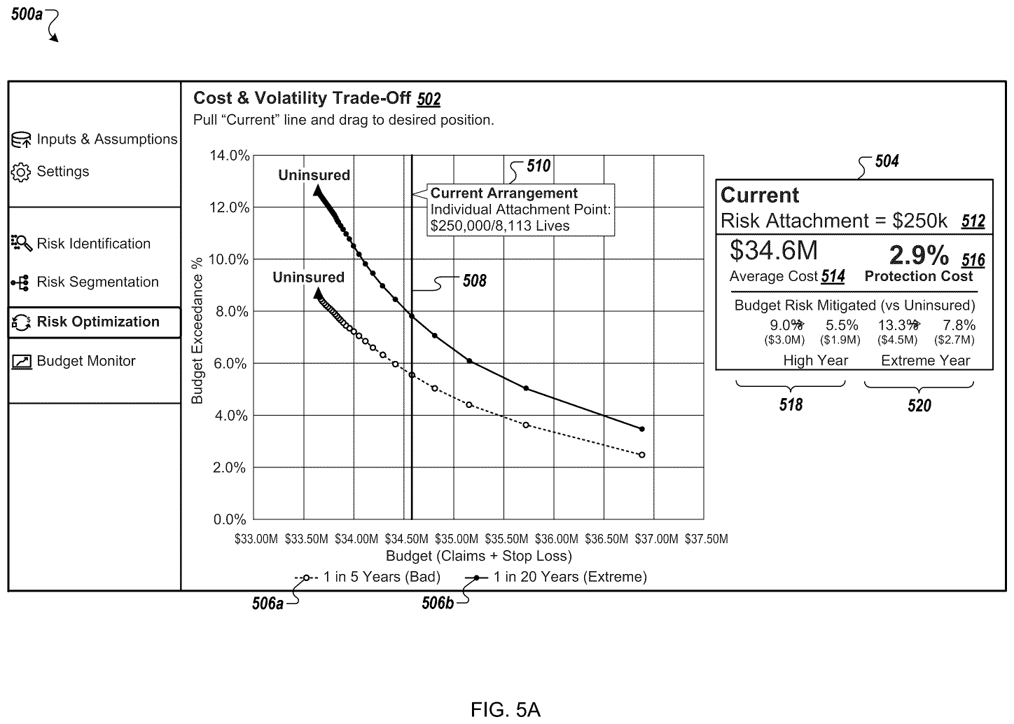

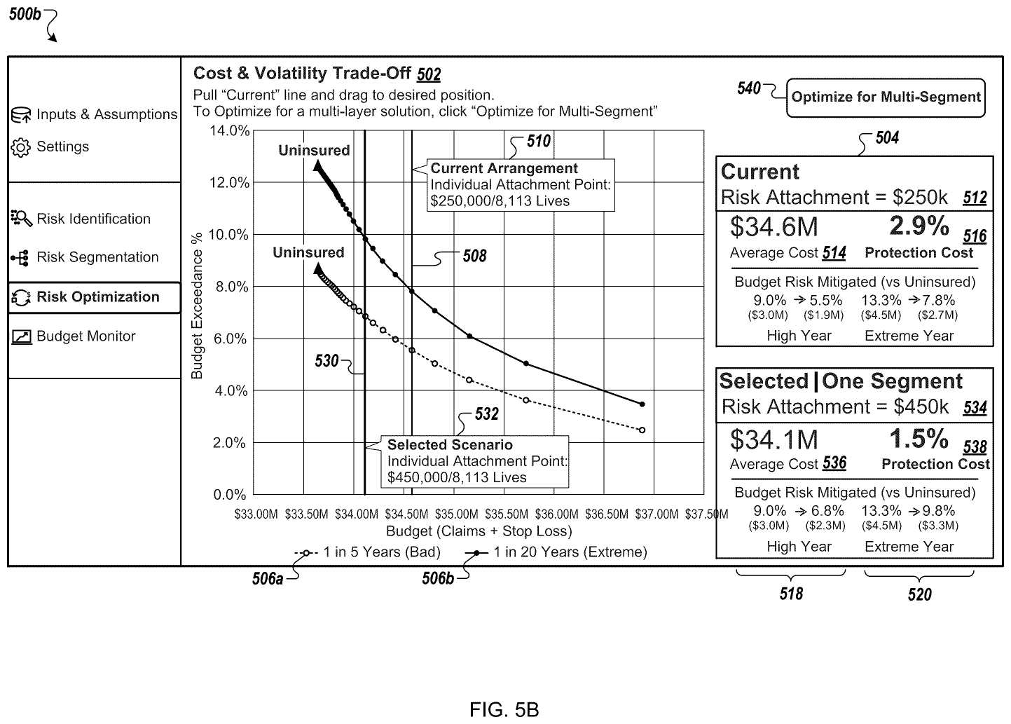

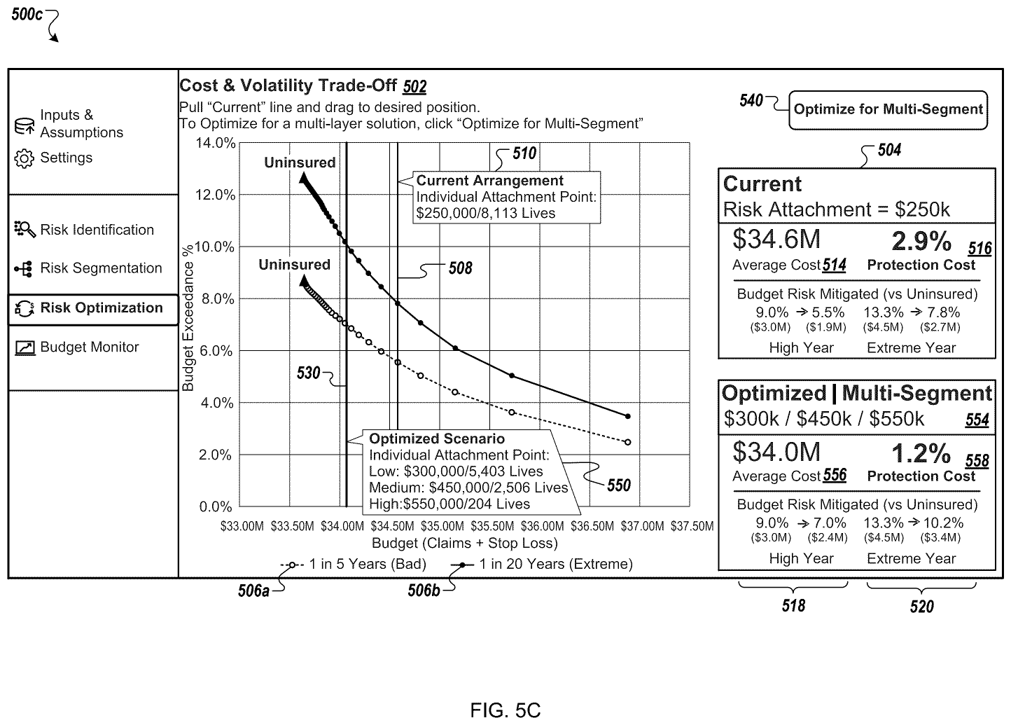

FIG. 5A through FIG. 5C illustrate a set of example graphical user interface screens 500a-c for presenting risk optimization trade-offs to alter outcomes predicted in the health risk analysis. The graphical user interface screens 500a-c, for example, present risk mitigation options for reinsurance positions in a cost and volatility trade-off graph 502 presented as budget vs. percentage budget exceedance. Two plots 506 are presented: a 1 in 5 years (bad) forecast plot 506a, and a 1 in 20 years (extreme) forecast plot 506b. A vertical line representing a current arrangement 508 intersects the two plots 506 to demonstrate the current position of the client with respect to risk mitigation. Adjacent to the graph 502, a trade-off analysis pane 504 presents forecast budget implications to electing various selected options (e.g., the current arrangement 508 or another option selected through moving the line representing the current location to another position along the x-axis of the graph 502).

Turning to FIG. 5A, a first GUI screen 500a presents a pop-up message 510 adjacent to the current arrangement indicating that the line represents an individual attachment point at $250,000 to cover 8,113 members of a population. In the trade-off analysis pane 504, details regarding the current arrangement 508 are listed, including a risk attachment value 512 (i.e., $250,000), an average cost 514 for health expenditures for the member population (i.e., $34.6 million), and a protection cost 516 (i.e., 2.9%). Further, an estimation of budget risk mitigated by the current arrangement 508 in comparison to a budget risk remaining uninsured is presented in relation to both a high year 518 (e.g., corresponding to the bad forecast plot 506a) and an extreme year 520 (e.g., corresponding to the extreme forecast plot 506b). As shown, based on the high year 518 risk transfer analysis, the current arrangement 508 is anticipated to leave 5.5% or $1.9 million uninsured as compared to 9.0% of the budget ($3.0 million) that would be uninsured without the reinsurance. Additionally, in the extreme year 520 risk transfer analysis, the current arrangement 508 is anticipated to leave 7.8% or $2.7 million of the budget uninsured as compared to 13.3% or $4.5 million of the budget that would remain uninsured without the reinsurance.

Turning to FIG. 5B, a second GUI screen 500b illustrates a situation including a selected scenario 530 which, as identified by a second pop-up message 532, represents an individual attachment point of $450,000 to cover the 8,113 members of the population. A second risk attachment value 534 of $450,000, corresponding to the selected scenario 530, has been added to the trade-off analysis pane 504. The second risk attachment value 534 is estimated to incur a $34.1 million average cost 536 for health expenditures for the member population and a protection cost 538 of 1.5%. An estimation of budget risk mitigated by the selected arrangement 530 in comparison to a budget risk remaining uninsured is presented in relation to both the high year 518 and the extreme year 520. As shown, based on the high year 518 risk transfer analysis, the selected arrangement 530 is anticipated to leave 6.8% or $2.3 million uninsured (as compared to $3.0 million or 9%). Additionally, in the extreme year 520 risk transfer analysis, the selected arrangement 530 is anticipated to leave 9.8% or $3.3 million of the budget uninsured (e.g., as compared to 13.3% or $4.5 million of the budget uninsured).

In some implementations, when a user moves the current arrangement line to select an optional scenario for review, a multi-segment optimization control 540 is presented for converting the selected risk attachment value into an optimized multi-layer solution where different attachment points correspond to different portions of the member population. Turning to FIG. 5C, a third GUI screen 500c represents the results of clicking the multi-segment optimization control 540 in the second GUI screen 500b of FIG. 5B. As illustrated in FIG. 5C, a third pop-up message presenting an optimized scenario overview 550 has replaced the second pop-up message 532, now detailing an optimized scenario in which individual attachment points are split between portions of the member population. The optimized scenario overview 550 states that, in the optimized scenario, a low attachment point of $300,000 corresponds to 5,403 members, a medium attachment point of $450,000 corresponds to 2,506 members, and a high attachment point of $550,000 corresponds to 204 members.

Turning to the trade-off analysis pane 504, an optimized multi-segment $300 k/$450 k/$550 k scenario 554 is presented, estimated to incur a $34.0 million average cost 556 for health expenditures for the member population and a protection cost 558 of 1.2%. An estimation of budget risk mitigated by the optimized scenario represented in the optimized scenario overview 550 in comparison to a budget risk remaining uninsured is presented in relation to both the high year 518 and the extreme year 520. As shown, based on the high year 518 risk transfer analysis, the optimized scenario represented by the optimized scenario overview 550 is anticipated to leave 7.0% or $2.4 million uninsured as compared to 9.0% of the budget ($3.0 million) uninsured without reinsurance. Additionally, in the extreme year 520 risk transfer analysis, the optimized scenario represented in the optimized scenario overview 550 is anticipated to leave 10.2% or $3.4 million of the budget uninsured, as compared to 13.3% or $4.5 million of the budget without application of reinsurance.

Returning to FIG. 1, in some implementations, the risk transfer analysis engine 134 pre-calculates optimized attachment scenarios such as the optimized scenario represented in the optimized scenario overview 550 of FIG. 5C based on the risk analysis performed on the member population by the health risk analysis platform 102. The pre-calculation may result in over a million possible combinations using each risk segmented group (e.g., as determined by the risk pattern identification engine 116 and dimensionally reduced by the dimensionality reduction engine 118) over the span of every possible attachment point. Further, in some embodiments, the optimized attachment scenarios include multi-layered approaches including multiple attachment points. The multiple attachment points, in some examples, may model a multi-layered approach, such as approaches involving internal pooling or captives. Upon user request (e.g., selection of the multi-segment optimization control 540 of FIG. 5B), a set of optimized scenario calculations are performed by the risk transfer analysis engine 134 to optimize at the attachment point(s) selected by the user (e.g., the selected arrangement 530 of FIG. 5B). The calculations, for example, may analyze aggregate expected cost as well as budget risk to optimize benefit to the client. The optimized scenarios, for example, may be identified through finding lowest cost, lowest risk, and/or a balanced combination of low cost and low risk.

In some embodiments, the risk transfer analysis engine 134 calculates the estimated effect one or more alternative financing arrangements may have on a client's risk mitigation position. For example, as illustrated in each of FIG. 4A and FIG. 4B, a distribution of individual paid claims 412a, b is illustrated as being distributed between a retained risk portion 414a, an internal pooling portion 414b (none used in the illustrated scenario), and a risk transfer portion 414c.

As an additional option for risk mitigation, in some implementations, a risk financing engine 136 calculates the potential impact of replacing group-level reinsurance parameters a risk financing solution involving one or more alternative financing arrangements. The alternative financing arrangements, in some examples, may include internal pooling, captives, investment funds, and/or credit financing. In some embodiments, the risk financing engine 136 analyzes the estimated impact on a client's budget volatility over the course of one or more time periods (e.g., monthly, quarterly, annual, year-over-year, etc.) (e.g., based on calculations performed by the volatility engine 138) as well as on the overall annual cost to the client 104. In an illustrative example involving a given client with a current stop loss policy set at $500,000, the risk financing engine 136 may calculate the estimated financial impact of replacing the stop loss policy with a line of credit to draw from for members who surpass the $500,000 threshold.

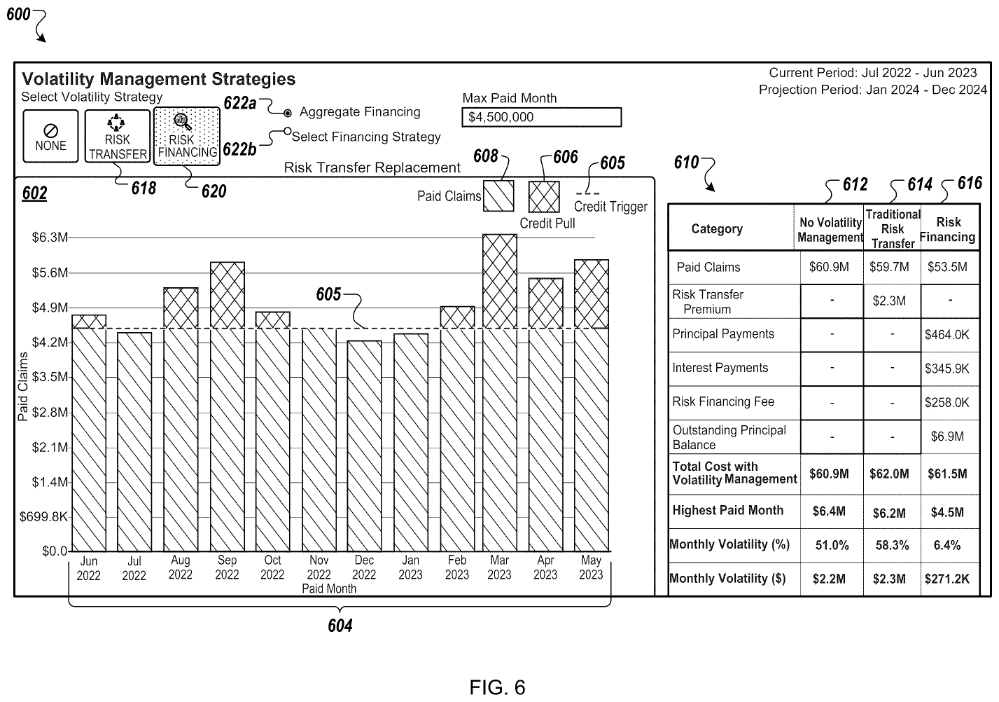

Turning to FIG. 6, a screen shot of an example risk financing user interface (UI) 600 illustrates a proposed credit financing option derived from calculations performed by the risk financing engine 136. The risk financing UI 600, for example, includes a graph section 602 presenting a series of bars representing forecasted monthly expenses 604 (e.g., corresponding to an average cost such as the average cost 514 of FIG. 5A, adjusted based on periodic volatility estimates generated by the volatility engine 138 of FIG. 1), a line graph of a credit trigger 605 representing a proposed credit financing option, a lower portion 608 of the bars 604 beneath or meeting the credit trigger line 605, and an upper portion 606 of certain bars 604 representing a credit pull where monthly expenses exceed the credit trigger 605.

Turning to a cost breakdown pane 610, a cost analysis of a forecast effect of risk financing on the client's budget is included. The forecast includes data representing an anticipated financial outcome with no volatility management 612, data representing a financial outcome with traditional risk transfer 614 applied, and data representing an anticipated financial outcome with risk financing 616 applied. A client may analyze the information presented in the risk financing user interface (UI) 600 to determine whether credit financing is a desirable option for the organization.

In interacting with the user interface 600, in some implementations, a client may evaluate options for structuring risk transfer (via a control 618) and/or risk financing (via a control 620). Further, the user interface 600 may be used to determine rules and/or triggers to guide the client in making decisions on when to trigger form(s) of risk financing based on a type of financing strategy being presented (e.g., an aggregate financing strategy 622a or a risk transfer replacement strategy 622b).

In some implementations, a report generation engine 142 combines data outputs from various engines of the health risk analysis platform 102, such as the resource forecasting analysis engine 124, the budget forecasting analysis engine 126, the benefits adjustment engine 132, the risk transfer analysis engine 134, the risk financing engine 136, the volatility engine 138, and/or the risk metrics engine 140 to produce insights useful to the clients 104. The insights, for example, may include static (printed/electronic file) reports, spreadsheets, online reports, and/or interactive web applications. The insights, in some examples, may be presented as charts, graphs, tables, and/or other performance indicators. In particular, the report generation engine 142 may produce the interactive web interfaces described in relation to FIG. 3, FIG. 4A, FIG. 4B, and FIG. 5A through FIG. 5C.

In some implementations, a risk mitigation benchmark engine 143 calculates, over a large pool of individuals (e.g., the members of all clients 104, all individuals contributing to the claims data 144 used by the risk pattern identification engine 116, etc.) benchmark metrics for comparison to the member population of a subject client 104. The risk mitigation benchmark engine 143, in some embodiments, generates benchmark metrics based on peer matching between the subject client and other member populations. For example, a member population in one geographic area (e.g., southern USA) may be compared to other member populations in the same region. In another example, a member population of a type of client (e.g., a trade organization, a corporation, an educational institution, a medical institution, etc.) may be benchmarked against member populations corresponding to the same type of client. The benchmark metrics, in some examples, may include a percentage of high risk members within the population, a percentage of members in each of the top N (e.g., 3, 5) high risk medical condition categories, and/or a predicted percentage of resources devoted to covering high risk members. The example GUI screen 400 of FIG. 4A, for example, illustrates a comparison of percentage of high risk members within the population 416a (4.4%) to a benchmark 416b (3.9%) as well as a comparison of predicted percentage of claims cost devoted to covering high risk members within the member population 418a (43.3%) to a benchmark population 418b (43.2%).

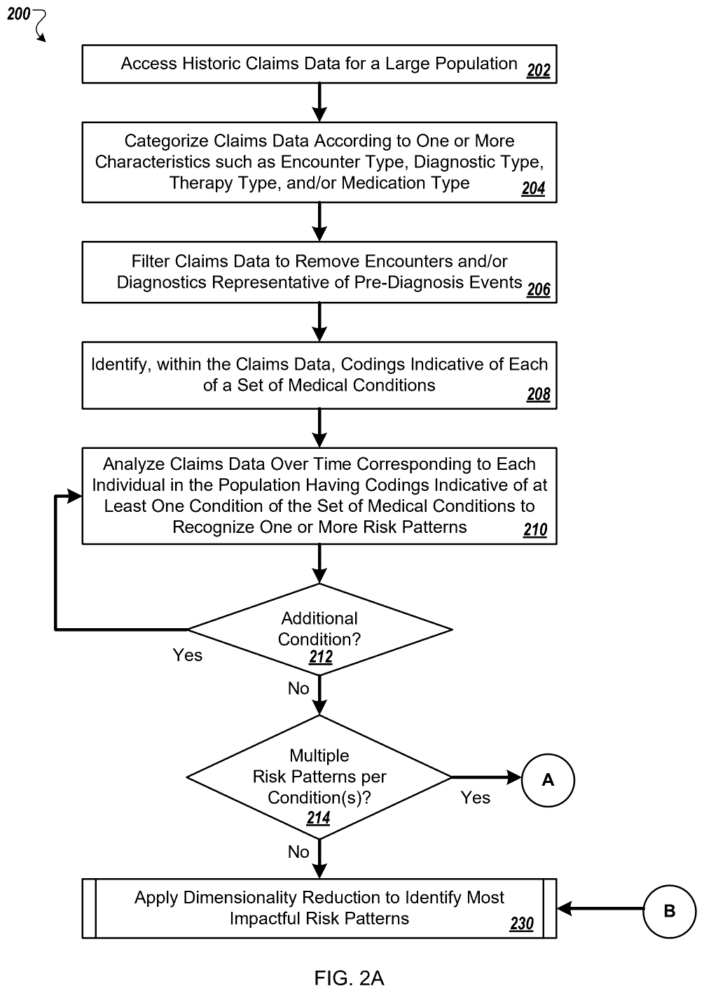

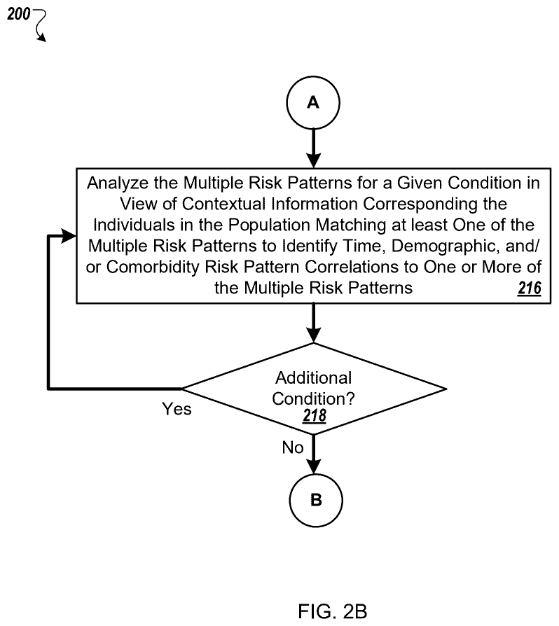

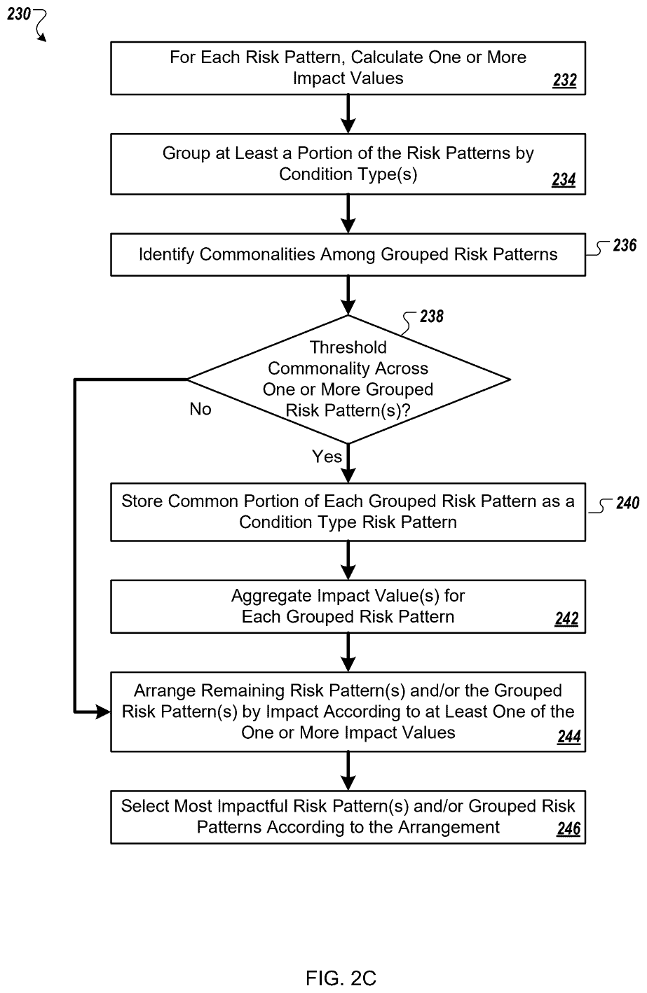

Turning to FIG. 2A and FIG. 2B, a flow chart illustrates an example method 200 for extracting risk patterns from insurance claims data. The method 200, for example, may be performed by portions of the health risk analysis platform 102 of FIG. 1, such as the member claims analysis engine 114 and/or risk pattern identification engine 116. The method 200, for example, may analyze claims data 144 obtained from the data repository 110 of FIG. 1 to derive risk patterns associated with one or more medical conditions.

In some implementations, the method 200 begins with accessing historic claims data for a large population (202). The claims data, for example, may be associated with de-identified patient demographic information (e.g., age or age range, gender, geographic region, etc.) for a large number of individuals. The demographic information, for example, may be stored as demographic data records. The individuals, for example, may be associated with the clients 104 of FIG. 1. The large number, for example, may be tens of thousands, hundreds of thousands, or a million or more patients. The historic claims data may span a time range of at least six months, at least one year, one to three years, up to five years, or longer than five years. To identify risk patterns, for example, an adequate timeframe for deriving a series of encounters and/or prescriptions associated with one or more medical conditions may be reviewed. For this reason, timeframes of less than six months or even less than one year may be less useful since patterns materializing over a longer timeframe may not be identified with a smaller data set.

In some implementations, the claims data is categorized according to characteristics of each claim such as, in some examples, an encounter type (e.g., inpatient, outpatient, professional, etc.), a diagnostic type (e.g., laboratory screenings, imaging screenings, etc.), a therapy type (e.g., rehabilitation/physical therapy, etc.) and/or a medication type (e.g., prescription, over the counter, etc.) (204). The categorizations, for example, may be determined based at least in part on one or more codes associated with each claim. The risk pattern identification engine 116 of FIG. 1, for example, may perform the categorization of the claims data.

In some implementations, the claims data is filtered to remove encounters and/or diagnostics representative of pre-diagnosis events (206). The claims may be analyzed in view of one or more codes, for example, to identify types of encounters and/or diagnostics indicative of pre-diagnosis events. In some embodiments, associated claims may be reviewed to categorize one or more encounters and/or diagnostics as being pre-diagnosis events. For example, common sets of laboratory diagnostics may be indicative of pre-diagnosis research into the cause for a patient's symptoms, while a same one or small number of laboratory diagnostics from among the common set may be performed as part of a post-diagnosis event. Further, patterns of encounters and/or diagnostics over time, such as periodic checks and/or diagnostics scheduled a certain period of time prior to a type of encounter, may be indicative of a post-diagnostic event rather than a pre-diagnostic event. Patterns in claims within windows of time (e.g., one day, three days, one week, two weeks, one month, etc.), in some embodiments, are analyzed to identify patterns of encounters and/or diagnostics representative of post-diagnostic events as opposed to pre-diagnostic events. The risk pattern identification engine 116 of FIG. 1, for example, may perform the filtering of the claims data.

In some implementations, codings are identified within the claims data indicative of each of a set of medical conditions (208). The codings, for example, may include particular medications, particular surgical encounters, particular therapy encounters, and/or particular prescription supplies (e.g., diagnostic strips, insulin, etc.) commonly associated with treating a medical condition or type of medical condition. The medical conditions, in some examples, can include one or more chronic conditions, acute conditions, diseases, and/or disorders. Further, the codings may include sets of codings of one or more of medications, encounters, and/or diagnostics corresponding to a particular medical condition. The codings, for example, may be analyzed by the risk pattern identification engine 116 of FIG. 1.

In some implementations, for each of the set of medical conditions (212), the claims data corresponding to those individuals in the population having codings indicative of the respective medical condition is analyzed over time to recognize one or more risk patterns within the claims data (210). Machine learning analysis, for example, may be performed to derive patterns within the claims data of each set of individuals corresponding to each medical condition. The risk pattern identification engine 116 of FIG. 1, for example, may perform the risk pattern analysis.

In some implementations, if the analysis derived two or more risk patterns corresponding to a particular medical condition (214), turning to FIG. 2B, the multiple risk patterns are analyzed in view of contextual information corresponding to the individuals of the population matching at least one of the multiple risk patterns to identify time, demographic, and/or comorbidity associations to one or more of the multiple risk patterns (216). For example, certain patterns may have a positive correlation to one or more of gender, age, race, and/or pre-existing condition(s). The risk pattern identification engine 116 of FIG. 1, for example, may perform the risk pattern correlation analysis. The risk pattern correlation analysis may be repeated (218) for each medical condition and/or medical condition type associated with multiple risk patterns.

Returning to FIG. 2A, in some implementations, once all medical conditions and/or medical condition types having multiple risk patterns associated with them have been analyzed (214), dimensionality reduction is applied to identify the most impactful risk patterns (230). The dimensionality reduction may be performed, for example, as described in relation to a method 230 of FIG. 2C. The dimensionality reduction engine 118 of FIG. 1 may perform the dimensionality reduction.

Although described in relation to a particular series of operations, in other embodiments, the method 200 may be performed in a different manner. In some implementations, more or fewer operations may be included in the method 200. For example, in some implementations, the risk patterns for each medical condition are analyzed (216) iteratively in view of multiple levels of demographic groupings/ranges and/or comorbidities/comorbidity groupings. In some implementations, one or more of the operations of the method 200 may be performed in a different order and/or concurrently. For example, the claims data may be filtered (206) prior to categorizing (204). Other modifications to the method 200 are possible.

FIG. 2C illustrates a flow chart of the example method 230 for applying dimensionality reduction to identify a set of the most impactful risk patterns from a large number of risk patterns. The risk patterns, for example, may be identified as described in relation to the risk pattern identification engine 116 of FIG. 1 and/or the method 200 of FIG. 2A and FIG. 2B. The dimensionality reduction engine 118 of FIG. 1, for example, may perform at least a portion of the method 230.

In some implementations, the method 230 begins with calculating, for each risk pattern, one or more impact values (232). The impact value(s), for example, may relate to resource allocation and/or severity of outcome. In some illustrative examples, the impact value(s) may include a mortality rate, a permanent disability rate, an insurance cost, an estimation of professional-hours devoted to care, a hospital bed count, an estimated difference in cost between prompt care and delayed care, and/or an estimated difference in mortality rate and/or permanent disability rate between prompt care and delayed care.

In some implementations, at least a portion of the risk patterns are grouped by medical condition type(s) (234). Certain risk patterns may pertain to similar and/or related conditions such that, rather than tracking thousands of individual risk patterns, each specific to a particular condition, closely related medical conditions may be grouped consideration as a “type” (e.g., pulmonary type, vascular type, etc.). The risk patterns may be grouped, for example, according to medical coding (e.g., each diabetes code may be nested in a hierarchy under a general diabetes category, etc.). The medical condition grouping engine 122 of FIG. 1, for example, may be used to identify medical condition groupings.

In some implementations, commonalities among grouped risk patterns are identified (236). The commonalities, for example, may include similar treatment patterns, common medications, common diagnostics, and/or common therapies. The commonalities, for example, may be identified through machine learning or artificial intelligence algorithms to identify closely matching patterns.

In some implementations, if a threshold level of commonality is identified across each of one or more grouped risk patterns (238), the common portion of each of these grouped risk patterns may be stored as a more general medical condition type risk pattern (240). The threshold level, for example, may include a threshold percentage of overlapping elements of the risk pattern and/or a threshold number of overlapping elements of the risk pattern. The elements, in some illustrative examples, may include a class or family of medication, a type of surgery, and/or a pattern of professional encounters.

In some implementations, the impact value(s) for each grouped risk pattern are aggregated (242). For example, the costs across all risk patterns contributing to the group risk pattern may be summed.

In some implementations, the remaining (non-grouped) risk patterns, and/or the grouped risk patterns are arranged by impact according to at least one of the one or more impact values (244). The risk patterns, for example, may be ranked from greatest cost to lowest cost. In another example, multiple impact values may be combined according to an impact equation. Various impact values may be weighted within the equation (e.g., a higher weight to higher mortality risk patterns).

In some implementations, the most impactful risk patterns and/or grouped risk patterns are selected according to the arrangement (246). As illustrated in FIG. 3, for example, the top 5 risks 302 according to estimated cost may be selected.

Although described in relation to a particular series of operations, in other embodiments, the method 230 may be performed in a different manner. In some implementations, more or fewer operations may be included in the method 230. For example, in some implementations, rather than grouping risk patterns (234) and determining whether a threshold commonality exists (238), machine learning or artificial intelligence may be used to group risk patterns based on having substantial commonality in pattern contents. In some implementations, one or more of the operations of the method 230 may be performed in a different order and/or concurrently. For example, in some implementations, the one or more impact values are calculated (232) after creating any grouped risk patterns (238-242). Other modifications to the method 230 are possible.

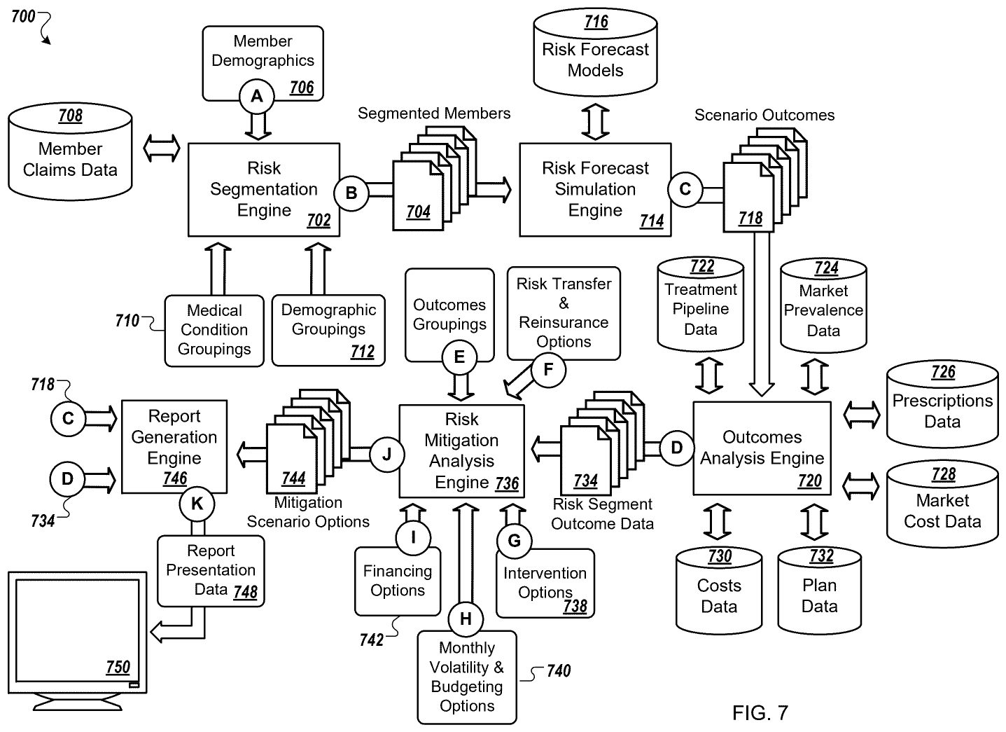

Turning to FIG. 7, an operational flow diagram of an example process 700 for analyzing claims data of a member population in view of health risks is illustrated. Portions of the process 700, for example, may be performed by the health risk analysis platform 102 of FIG. 1. The process 700, for example, may be used to generate the graphical user interface displays described in relation to FIG. 3, FIG. 4A, FIG. 4B, FIG. 5A through FIG. 5C, and/or FIG. 6.

In some implementations, the process 700 begins with a risk segmentation engine 702 segmenting a member population into subsets of segmented members 704. The risk segmentation engine 702, for example, may be the risk segmentation engine 128 of FIG. 1. The member population, for example, may be identified by member demographics 706 which can be cross-referenced with a set of member claims data 708. The risk segmentation engine 702, in some embodiments, uses the member demographics information 706 to divide the members into asset of demographic groupings. The demographic groupings, for example, may include predetermined demographic groupings 712. In another example, certain demographic groupings may be accomplished by the risk segmentation engine 702 in accordance with medical conditions identified via the member claims data 708. In illustration, certain age ranges may be pertinent to particular medical conditions. In some embodiments, the risk segmentation engine 702 uses the member claims data 708 to divide the members into a set of medical condition groupings 710. The risk segmentation engine 702, for example, may use a set of predetermined medical condition groupings 710 for dividing the members into the medical condition groupings. In another example, the risk segmentation engine 702 may determine medical condition groupings based on numbers of members exhibiting each of a set of conditions. For example, if one or very few members are identified as having a specific condition, that condition may be grouped with other specific conditions into a more general condition type for risk evaluation purposes.

In some implementations, the subsets of segmented members 704 are provided to a risk forecast simulation engine 714. The risk forecast simulation engine, for example, may perform various operations described in relation to the resource forecasting analysis engine 124 and/or the budget forecasting analysis engine 126 of FIG. 1. The risk forecast simulation engine 714, in some embodiments, applies a set of risk forecast models 716 to the subsets of segmented members 704 to generate a set of scenario outcomes 718 representing simulated results (e.g., health outcomes, costs, resource allocations that will be used, etc.) in treating the member population based on their medical condition groupings and/or demographic groupings in view of the risk forecast models 716. In other words, the set of scenario outcomes 718 may include outcomes for each risk segment analyzed by the risk forecast simulation engine 714. The risk forecast models 716, for example, may represent analysis performed by the risk pattern identification engine 116 in analyzing a large number of individuals in view of a time period of claims data to quantify likely outcomes (e.g., costs, health results, resource needs, etc.) in view of a target member population's particular demographics and/or medical conditions. The scenario outcomes 718 can include various time frames for outcome analysis (e.g., a subset of monthly scenarios covering a span of one or more months, a subset of six-month scenarios, a subset of one year scenarios, etc.). Further, the scenario outcomes 718 may encompass various magnitudes of simulation outcome (e.g., probable outcome, a “bad year” outcome, and an “extreme year” outcome as described in relation to the budget risk estimates of FIG. 5B). The scenarios 718, for example, may include numbers and/or timings of various encounters for each of the subsets of member segments 704 according to each risk forecast model 716 applicable to that subset. Further, the scenarios 718 may include medications, medical equipment, and/or staffing/clinical resource needs for each of the subsets of member segments 704 according to each risk forecast model 716 applicable to that subset.

In some implementations, an outcomes analysis engine 720 applies one or more sets of quantifying data to the scenario outcomes 718 to quantify resource needs and/or health outcomes in view of a particular client. The outcomes analysis engine 720, for example, may perform a portion of the operations described in relation to the outcomes analysis engine 120 of FIG. 1.

The outcomes analysis engine 720, in some embodiments, maps the generalized scenario outcomes 718 to particular treatment pipeline data 722 representing the therapeutic process(es) typical to the member population. The treatment pipeline data 722, for example, may represent therapeutic process(es) adopted by a particular clinic or medical facility overseeing treatment of the member population. In another example, the treatment pipeline data 722 may represent common therapeutic process(es) to a geographic region in which the member population is based. Further, the treatment pipeline data 722 may represent typical timing of encounters (e.g., for the region, for the particular clinic or medical facility, etc.) which may differ from the underlying assumptions applied in the scenario outcomes 718.

In some embodiments, the outcomes analysis engine 720 maps the generalized scenario outcomes 718 to market prevalence data 724 representing typical disease prevalence and/or progression to the market of the target member population. The market prevalence data 724, for example, may weigh the frequencies of subsets of scenario outcomes based on what may be typical to the geographic region of the subject member population and/or a subject medical facility.

The outcomes analysis engine 720, in some embodiments, maps the scenario outcomes 718 to prescriptions data 726 to determine stocking requirements and/or compounding requirements based on the member population. The prescriptions data 726, for example, may include availability of medical equipment, medicines, and/or other consumables related to the scenario outcomes 718. The prescriptions data 726 may include information such as lead times for ordering, bulk purchase information (e.g., quantities available), and/or combination packaging available (e.g., certain equipment/medications may be sold in sets of items commonly used together).

In some embodiments, the outcomes analysis engine 720 maps the scenario outcomes 718 to market cost data 728 to quantify the scenario outcomes 718 in light of market costs applicable to the member population (or client thereof). For example, the outcomes analysis engine 720 may map numbers of each type of hospital bed to cost per those types of hospital beds. In another example, the outcomes analysis engine 720 may map staffing requirements to staffing costs for ensuring appropriate professional support to the member population.

The outcomes analysis engine 720, in some embodiments, maps the scenario outcomes 718 to plan data 732 to quantify the encounters, prescriptions, etc. in view of insurance plan coverage. The plan data 732 may include a variety of plan options elected by various members of the population, such that distributions of plan options may be applied by the outcomes analysis engine 720 to estimate usage in view of coverage distributions across the member population.

In some embodiments, the outcomes analysis engine 720 maps the scenario outcomes 718 to costs data 730. The costs data 730, for example, may include costs based on insurance coverage (e.g., in view of the plan data 732). Further, the costs data 730 may include costs for staffing, medications, and other resources identified in the scenario outcomes 718 and/or based on the treatment pipeline data 722 mappings, the prescriptions data mappings 726, and/or the market cost data mappings 728.

In some implementations, the outcomes analysis engine 720 provides sets of risk segment outcome data 734 (e.g., costs per risk segment, insurance plan usage per risk segment, prescription needs per risk segment, treatment requirements per risk segment, etc.) to a risk mitigation analysis engine 736. As with the scenario outcomes 718, the risk segment outcome data 734 may include individual subsets of data directed to a most likely scenario (e.g., typical year), a conservative scenario (e.g., a bad year), and/or a worst case scenario (e.g., an extreme year).

The risk mitigation analysis engine 736, in some implementations, analyzes the risk segment outcome data 734 in view of intervention options 738, monthly volatility and budgeting options 740, and/or financing options 742 to develop a set of mitigation scenario options 744. The mitigation scenario options 744, unlike the scenario outcomes 718 and the risk segment outcome data 734, may provide an overall view of the subject client's forecast circumstances for the upcoming time period in view of the member population's member demographics 706 and/or member claims data 708. The risk mitigation analysis engine 736, for example, may provide, in the mitigation scenario options 744, solutions to at least partially mitigate the risk to the client of being overwhelmed with resource and/or budgeting needs. Further, the mitigation scenario options 744 may propose priorities for client focus, such as members of the population exhibiting particular risk patterns of medical conditions, to improve upon likely negative health outcomes. The risk mitigation analysis engine 736, for example, may perform a portion of the operations described in relation to the risk prioritization engine 130, the risk transfer analysis engine 134, and/or the risk financing engine 136 of FIG. 1.

In some embodiments, the risk mitigation analysis engine 736 analyzes the risk segment outcome data 734 in view of a set of intervention options 738 for one or more medical conditions to anticipate potential benefit of focusing on a key portion of the member population in the greatest need of immediate medical support.

In some embodiments, the risk mitigation analysis engine 736 analyzes the risk segment outcome data 734 in view of monthly volatility and budgeting options 740 to propose periodic budget allocations for preparing the client for likely (or bad, or worst-case scenario, etc.) upcoming expenses. The volatility information, for example, may be derived as described in relation to the volatility engine 138 of FIG. 1.

The risk mitigation analysis engine 736, in some embodiments, analyzes the risk segment outcome data 734 in view of financing options 742 to propose additional and/or alternative sources for mitigating financial risk, such as the options described in relation to the risk financing engine 136 of FIG. 1.

In some implementations, a report generation engine 746 converts the mitigation scenario options 744, the scenario outcomes 718, and/or the risk segment outcome data 734 into report presentation data 748 for presenting actionable information to an end user via a display 750. The report generation engine 746, for example, may perform a portion of the operations as described in relation to the report generation engine 142 of FIG. 1. Further, the report generation engine 746 may perform a portion of the operations described in relation to the risk metrics engine 140, and/or the risk mitigation benchmark engine 143 of FIG. 1. For example, the report generation engine 746 may generate the user interface displays described in relation to FIG. 3, FIG. 4A, FIG. 4B, FIG. 5A through FIG. 5C, and/or FIG. 6. The report, in some embodiments, includes interactive content, allowing the end user to filter, drill-down, and/or rearrange the information presented.

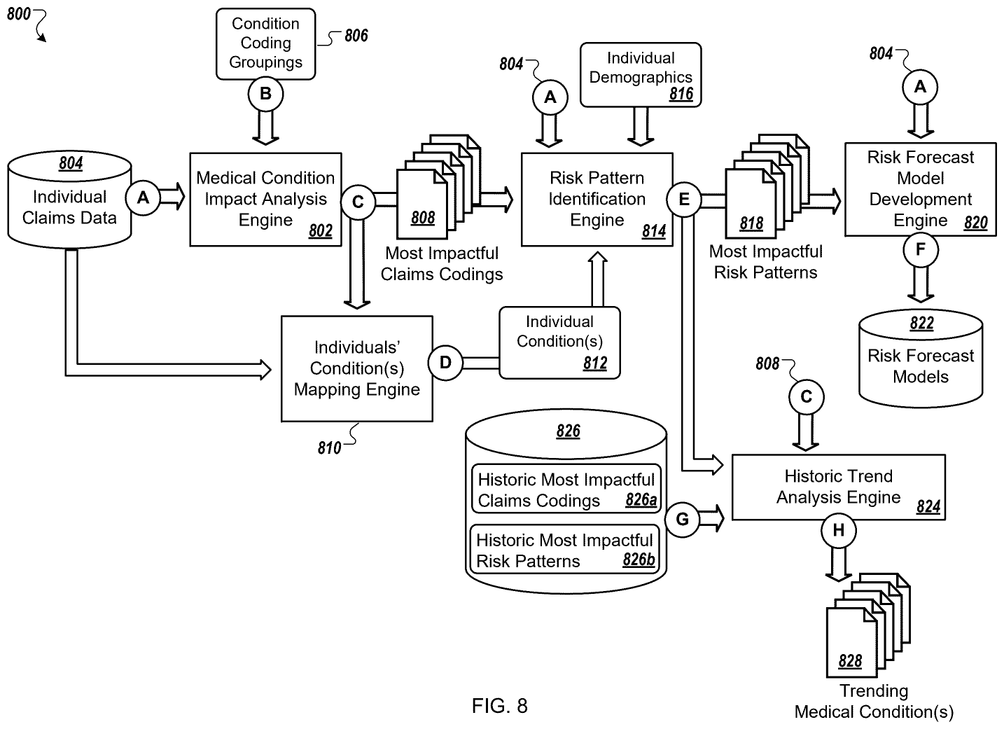

Turning to FIG. 8, a flow diagram of an example process 800 for generating risk forecast models for use in the process of FIG. 7 is presented. The process 800, for example, may be performed by the health risk analysis platform 102 to analyze the claims data 144 to identify risk patterns.

In some implementations, the process 800 begins with a medical condition impact analysis engine 802 analyzing individual claims data 804 of a large pool of individuals to identify the most impactful claims codings 808. To perform the analysis, in some embodiments, the medical condition impact analysis engine 802 maps the individual claims data 804 to condition coding groupings 806 to identify claims directed to a same medical condition/condition type whose treatment encounters and/or prescription medications were coded in a slightly different manner. For example, a generic form of a diabetes treatment may be coded differently than a brand name form of the diabetes treatment while covering the same medical condition and having a similar effect. The medical condition impact analysis engine 802 may also filter out claims data unrelated to and/or not descriptive of a particular medical condition. The most impactful claims codings 808, for example, may be the most prevalent codings within the claims data 804 (e.g., the most common medical conditions among the pool of individuals). In some embodiments, the medical condition impact analysis engine 802 cross-references the claims codings to estimated costs related to each of a set of medical conditions to determine the most impactful claims codings 808.

In some implementations, an individual medical conditions mapping engine 810 cross-references the most impactful claims codings 808 with the individual claims data 804 to identify a subset of the pool of individuals exhibiting the most impactful medical conditions. The individual medical conditions mapping engine 810, in some embodiments, provides individual conditions information 812 for use by a risk pattern identification engine 814.

The risk pattern identification engine 814, in some implementations, evaluates a portion of the individual claims data 804 corresponding to the subset of individuals identified in the individual conditions information 812 to identify a set of most impactful risk patterns 818. The risk pattern identification engine 814, in some embodiments, evaluates the portion of the individual claims data 804 in view of individual demographics 816 corresponding to the subset of individuals identified in the individual conditions information 812 to identify risk patterns including demographic correlations. The risk pattern identification engine 814, for example, may perform a portion of the operations described in relation to the risk pattern identification engine 116 of FIG. 1. The risk pattern identification engine 814 may also perform a portion of the operations described in relation to the dimensionality reduction engine 118 of FIG. 1.

In some implementations, a risk forecast model development engine 820 analyzes the most impactful risk patterns 818 to develop a set of risk forecast models 822 for identifying, from claims data such as the individual claims data 804, risk patterns matching each of the most impactful risk patterns 818. The risk forecast models 822, for example, may include a separate model for each of the conditions corresponding to the most impactful claims codings 808. The risk forecast model development engine 820 may apply statistical analysis and/or machine learning classifiers to develop at least a portion of the risk forecast models 822. The risk forecast models 822, for example, may be trained using the individual claims data 804 to identify individuals at risk of the conditions corresponding to the most impactful claims codings 808. In some embodiments, the risk forecast models 822 include one or more machine learning models. The machine learning models may include different or the same types of machine learning classifiers, such as regularized regression decision tree and/or gradient classifiers designed to capture layers of granularity in the risk patterns 818 as expressed within the individual claims data 804.

The risk forecast model development engine 820, in some embodiments, develops one or more artificial intelligence models designed to analyze claims data of an individual to extract risk pattern(s) indicative of one or more of the most impactful conditions. The artificial intelligence model(s), in some examples, can include artificial intelligence neural networks, synaptic neural networks, and/or trained and/or tuned foundational model system(s).

In some implementations, the most impactful risk patterns 818 and/or the most impactful claims codings 808 are also used by a historic trend analysis engine 824 to identify trending medical conditions 828 in view of historic data 826 (e.g., historic most impactful claims codings 826a and/or historic most impactful risk patterns 826b). The historic trend analysis engine 824, for example, may identify one or more medical conditions that are exhibiting a downward trend in prevalence. This may be due, for example, to a medical advancement such as a new vaccine, therapy, or screening technique to mitigate the effect of each medical condition exhibiting the downward trend. Further, the historic trend analysis engine 824 may identify one or more medical conditions that are new to the most impactful set of conditions or are otherwise increasing in prevalence. This could be due to greater awareness in the medical community, for example, leading to more accurate diagnosis of certain medical conditions. Further, an upward trend may be due to increased spread of a communicable disease. The trending medical conditions 828, for example, may be presented in a report for an end user to increase awareness of changing patient needs.

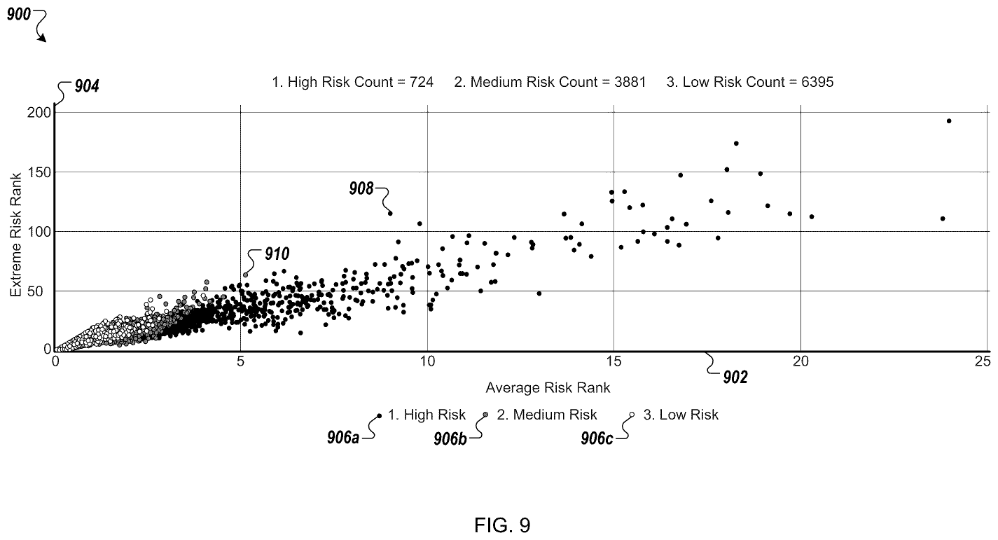

Since, as described for example in relation to FIG. 7, scenario outcomes representing simulated results may be generated according to a distribution of potential outcomes (e.g., magnitudes of simulation), the scenario outcomes may be further analyzed to identify target (e.g., high risk) members of a population according to outcome distribution (e.g., relative impact based on multiple simulation data points). FIG. 9 illustrates an example graphical presentation 900 representing members of a population in a scatter plot format according to both an average risk rank 902 (the x-axis) and an extreme risk rank 904 (the y-axis). The average risk rank 902, for example, may correspond to a “typical year” outcome, while the extreme risk rank 904 may correspond to an “extreme year” outcome (e.g., as described in relation to the budget risk estimates of FIG. 5B). The cost estimates used to calculate the extreme risk rank 904 for each member, in illustration, may represent the top 5% of worst outcomes calculated for that particular member. The average risk rank 902, for example, may be calculated by dividing the “typical” expected cost for each member by an average “typical” expected cost across the entire population. Further, the extreme risk rank 904 may be calculated by dividing the “extreme” expected cost for each member by an average “typical” expected cost across the entire population.

In the graphical presentation 900, the members of the population are divided into three risk categories: a high-risk category 906a (e.g., color-coded red); a medium risk category 906b (e.g., color-coded dark gray); and a low risk category 906c (e.g., color-coded light gray). As illustrated at a top of the graphical presentation 900, 724 members are categorized as high risk 906a, 3,881 members are categorized as medium risk 906b, and 6,395 members are categorized at low risk 906c. These risk categories, for example, may be calculated as described in relation to the risk categories 410 of FIG. 4A.

Additionally, for the purposes of the graphical presentation 900, members are scored by averaging the simulated cost outcomes produced on a per-member basis, thereby producing the “risk rank.” An average risk rank 902, for example, may be based on calculating an average of each individual member's overall simulated costs taking into account a large number of simulation results (e.g., tens, hundreds, or thousands of simulations, etc.). In another example, an extreme risk rank 904 may be based on calculating an average of each simulation for a given individual member having an anticipated cost above a threshold level (e.g., the 95th percentile). As can be seen, the potential “extreme” results can move some members not otherwise considered to be of high risk into a realm of potential greater risk.

The resultant scatter plot of the example graphical presentation 900 illustrates broad ranges of potential outcomes (e.g., from about 8 times the typical cost to about 120 times the typical cost for example member 908). By analyzing members according to multiple levels of risk, for example, those members exhibiting a potential for high volatility in outcome (e.g., an extreme risk rank 904 of 50 or greater in addition to an average risk rank 902 of 10 or greater) may be prioritized (e.g., in distributing limited resources for interventions, etc.). Further, members not predicted to be low risk may still demonstrate a relatively high extreme risk rank 904, which could result in consideration for inclusion in the prioritized members. In illustration, an example member 910, categorized as medium risk and having an average risk rank 902 of about 5.5, has an extreme risk rank of about 70, far higher than many high risk 906a members, including some high risk members having an average risk rank 902 double that of the example member 910.

Reference has been made to illustrations representing methods and systems according to implementations of this disclosure. Aspects thereof may be implemented by computer program instructions. These computer program instructions may be provided to a processor of a general-purpose computer, special purpose computer, or other programmable data processing apparatus and/or distributed processing systems having processing circuitry, such that the instructions, which execute via the processor of the computer or other programmable data processing apparatus, create means for implementing the functions/operations specified in the illustrations.

One or more processors can be utilized to implement various functions and/or algorithms described herein. Additionally, any functions and/or algorithms described herein can be performed upon one or more virtual processors. The virtual processors, for example, may be part of one or more physical computing systems such as a computer farm or a cloud drive.

Aspects of the present disclosure may be implemented by software logic, including machine readable instructions or commands for execution via processing circuitry. The software logic may also be referred to, in some examples, as machine readable code, software code, or programming instructions. The software logic, in certain embodiments, may be coded in runtime-executable commands and/or compiled as a machine-executable program or file. The software logic may be programmed in and/or compiled into a variety of coding languages or formats.

Aspects of the present disclosure may be implemented by hardware logic (where hardware logic naturally also includes any necessary signal wiring, memory elements and such), with such hardware logic able to operate without active software involvement beyond initial system configuration and any subsequent system reconfigurations (e.g., for different object schema dimensions). The hardware logic may be synthesized on a reprogrammable computing chip such as a field programmable gate array (FPGA) or other reconfigurable logic device. In addition, the hardware logic may be hard coded onto a custom microchip, such as an application-specific integrated circuit (ASIC). In other embodiments, software, stored as instructions to a non-transitory computer-readable medium such as a memory device, on-chip integrated memory unit, or other non-transitory computer-readable storage, may be used to perform at least portions of the herein described functionality.

Various aspects of the embodiments disclosed herein are performed on one or more computing devices, such as a laptop computer, tablet computer, mobile phone or other handheld computing device, or one or more servers. Such computing devices include processing circuitry embodied in one or more processors or logic chips, such as a central processing unit (CPU), graphics processing unit (GPU), field programmable gate array (FPGA), application-specific integrated circuit (ASIC), or programmable logic device (PLD). Further, the processing circuitry may be implemented as multiple processors cooperatively working in concert (e.g., in parallel) to perform the instructions of the inventive processes described above.

The process data and instructions used to perform various methods and algorithms derived herein may be stored in non-transitory (i.e., non-volatile) computer-readable medium or memory. The claimed advancements are not limited by the form of the computer-readable media on which the instructions of the inventive processes are stored. For example, the instructions may be stored on CDs, DVDs, in FLASH memory, RAM, ROM, PROM, EPROM, EEPROM, hard disk or any other information processing device with which the computing device communicates, such as a server or computer. The processing circuitry and stored instructions may enable the computing device to perform, in some examples, the method 200 of FIG. 2A and FIG. 2B, the method 230 of FIG. 2C, the process 700 of FIG. 8, and/or the process 800 of FIG. 8.

These computer program instructions can direct a computing device or other programmable data processing apparatus to function in a particular manner, such that the instructions stored in the computer-readable medium produce an article of manufacture including instruction means which implement the function/operation specified in the illustrated process flows.

Embodiments of the present description rely on network communications. As can be appreciated, the network can be a public network, such as the Internet, or a private network such as a local area network (LAN) or wide area network (WAN) network, or any combination thereof and can also include PSTN or ISDN sub-networks. The network can also be wired, such as an Ethernet network, and/or can be wireless such as a cellular network including EDGE, 3G, 4G, and 5G wireless cellular systems. The wireless network can also include Wi-Fi®, Bluetooth®, Zigbee®, or another wireless form of communication. The network, for example, may support communications between the health risk analysis platform 102 and each of the clients 104, the claims data source(s) 106, and/or the benefits coverage source(s) 108.

The computing device, in some embodiments, further includes a display controller for interfacing with a display, such as a built-in display or LCD monitor. A general purpose I/O interface of the computing device may interface with a keyboard, a hand-manipulated movement tracked I/O device (e.g., mouse, virtual reality glove, trackball, joystick, etc.), and/or touch screen panel or touch pad on or separate from the display. The display controller and display may enable presentation of the screen shots illustrated, in some examples, in FIG. 4A, FIG. 4B, FIG. 5A through FIG. 5C, FIG. 6, and/or FIG. 9.

Moreover, the present disclosure is not limited to the specific circuit elements described herein, nor is the present disclosure limited to the specific sizing and classification of these elements. For example, the skilled artisan will appreciate that the circuitry described herein may be adapted based on changes in battery sizing and chemistry or based on the requirements of the intended back-up load to be powered.

The functions and features described herein may also be executed by various distributed components of a system. For example, one or more processors may execute these system functions, where the processors are distributed across multiple components communicating in a network. The distributed components may include one or more client and server machines, which may share processing, in addition to various human interface and communication devices (e.g., display monitors, smart phones, tablets, personal digital assistants (PDAs)). The network may be a private network, such as a LAN or WAN, or may be a public network, such as the Internet. Input to the system, in some examples, may be received via direct user input and/or received remotely either in real-time or as a batch process.

Although provided for context, in other implementations, methods and logic flows described herein may be performed on modules or hardware not identical to those described. Accordingly, other implementations are within the scope that may be claimed.

In some implementations, a cloud computing environment, such as Google Cloud Platform™ or Amazon™ Web Services (AWS™), may be used perform at least portions of methods or algorithms detailed above. The processes associated with the methods described herein can be executed on a computation processor of a data center. The data center, for example, can also include an application processor that can be used as the interface with the systems described herein to receive data and output corresponding information. The cloud computing environment may also include one or more databases or other data storage, such as cloud storage and a query database. In some implementations, the cloud storage database, such as the Google™ Cloud Storage or Amazon™ Elastic File System (EFS™), may store processed and unprocessed data supplied by systems described herein. For example, the contents of the data repository 110 of FIG. 1, the member claims data 708, the treatment pipeline data 722, the market prevalence data 724, the prescriptions data 726, the market cost data 728, the costs data 730, and/or the plan data 732 of FIG. 7, and/or the individual claims data 804 and/or the historic data 826 of FIG. 8 may be maintained in a database structure.

The systems described herein may communicate with the cloud computing environment through a secure gateway. In some implementations, the secure gateway includes a database querying interface, such as the Google BigQuery™ platform or Amazon RDS™. The data querying interface, for example, may support access by the health risk analysis platform 102 to the claims data source(s) 106 and/or the benefits coverage source(s) 108.

In some implementations, an edge server is used to transfer data between one or more computing devices and a cloud computing environment according to various embodiments described herein. The edge server, for example, may be a computing device configured to execute processor intensive operations that are sometimes involved when executing machine learning processes, such as risk forecast simulation operations performed by the risk forecast simulation engine 714 of FIG. 7. An edge server may include, for example, one or more GPUs that are capable of efficiently executing matrix operations as well as substantial cache or other high-speed memory to service the GPUs. An edge server may be a standalone physical device. An edge server may be incorporated into other computing equipment, such as a laptop computer, tablet computer, medical device, or other specialized computing device. Alternatively or additionally, an edge server may be located within a carrying case for such computing equipment. An edge server, in a further example, may be incorporated into the communications and processing capabilities of a mobile unit such as a vehicle or drone, or may otherwise be located within the mobile unit.

In some implementations, the edge server communicates with one or more local devices to the edge server. The edge server, for example, can be used to move a portion of the computing capability traditionally shifted to a cloud computing environment into the local environment so that any computation intensive data processing and/or analytics required by the one or more local devices can run accurately and efficiently. In some embodiments, the edge server is used to support the one or more local devices in the absence of a connection with a remote computing environment. The edge server may be configured to communicate with the one or more local devices directly or via a network. For instance, the edge server can include a private wireless network interface, a public wireless network interface, and/or a wired interface through which the edge server can communicate with the one or more local devices. In some embodiments, certain local devices may be configured to communicate indirectly with the edge server, for example via another local device. Further, the edge server may be configured to communicate with a remote computing (e.g., cloud) environment via one or more public or private wireless network interfaces.

In some implementations, the method 200 of FIG. 2A and FIG. 2B, the method 230 of FIG. 2C, the process 700 of FIG. 7, and/or the process 800 of FIG. 8 may be configured to be performed in part by an edge server or a device interoperating with an edge server. The device interoperating with the edge server, for example, may share processing functionality with the edge server via one or more APIs implemented by the processes.

The systems described herein may include one or more artificial intelligence (AI) neural networks for performing automated analysis of data. The AI neural networks, in some examples, can include a synaptic neural network, a deep neural network, a transformer neural network, and/or a generative adversarial network (GAN). The AI neural networks may be trained using one or more machine learning techniques and/or classifiers such as, in some examples, anomaly detection, clustering, and/or supervised and/or association. In one example, the AI neural networks may be developed and/or based on a bidirectional encoder representations for transformers (BERT) model by Google of Mountain View, CA.

The systems described herein may communicate with one or more foundational model systems (e.g., artificial intelligence neural networks). The foundational model system(s), in some examples, may be developed, trained, tuned, fine-tuned, and/or prompt engineered to evaluate data inputs such as the claims data 144, the medical data 146, the demographics data 148, the medical condition groupings 150, and/or the demographic groupings 152 of the data repository 110 of FIG. 1, the claims data 708 member demographics data 706, medical condition groupings 710, demographic groupings 712, and/or segmented member data 704 of FIG. 7, and/or the individual claims data 804, the condition coding groupings 806, the impactful claims codings 808, the individual conditions 812, and/or the individual demographics 816 of FIG. 8. The foundational model systems, in some examples, may include or be based off of the generative pre-trained transformer (GPT) models available via the OpenAI platform by OpenAI of San Francisco, CA (e.g., GPT-3, GPT-3.5, and/or GPT-4) and/or the generative AI models available through Azure OpenAI or Vertex AI by Google of Mountain View, CA (e.g., PaLM 2).

Certain foundational models may be fine-tuned as AI models trained for performing particular tasks required by the systems described herein. Training material, for example, may be submitted to certain foundational models to adjust the training of the foundational model for performing types of analyses described herein.

Multiple foundational model systems may be applied by the systems and methods described herein depending on context. The context, for example, may include type(s) of data, type(s) of response output desired (e.g., at least one answer, at least one answer plus an explanation regarding the reasoning that lead to the answer(s), etc.). In another example, the context can include user-based context such as demographic information, entity information, and/or product information. In some embodiments, a single foundational model system may be dynamically adapted to different forms of analyses requested by the systems and methods described herein using prompt engineering.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

What is claimed is:

1. A system for deriving risk patterns corresponding to a set of medical conditions through analyzing historic treatment of a population of individuals, the system comprising:
a non-transitory computer readable storage region comprising one or more computer readable storage devices, the non-transitory computer readable storage region being configured to store
a plurality of treatment data records collected over at least a threshold period of time,
a plurality of demographic data records comprising demographic information for each member of a plurality of members of a population, wherein each record of the plurality of demographic data records correlates to a corresponding portion of the plurality of treatment data records, and
a plurality of risk forecast models trained, using historic claims data corresponding to a large population of individuals, to predict a likelihood of each of a plurality of medical conditions based on treatment data analysis, each risk forecast model configured to identify a respective at least one risk pattern corresponding to a respective at least one medical condition of the plurality of medical conditions; and
processing circuitry configured to perform operations comprising
accessing the plurality of treatment data records and the plurality of demographic data records,
segmenting the plurality of treatment data records into sets of segmented treatment data records according at least to a plurality of demographic groupings,
for each respective risk forecast model of the plurality of risk forecast models, applying one or more sets of the segmented claims data to the respective risk forecast model to identify a portion of the plurality of members of the population represented in the one or more sets of the segmented claims data as a respective clinical population being likely to have or develop the respective at least one medical condition corresponding to the respective risk forecast model,
analyzing information corresponding to each respective clinical population to determine one or more resource forecasts for treating the portion of the plurality of members over at least one predetermined future time period, wherein the one or more forecast resources comprise at least one of an estimated cost, an estimated staffing need, an estimated equipment need, or an estimated clinical resource need, and
prioritizing, based on the one or more resource forecasts, at least one of the plurality of medical conditions or the portion of the plurality of members.

2. The system of claim 1, wherein the plurality of medical conditions comprises at least one of one or more diseases or one or more chronic disorders.

3. The system of claim 1, wherein at least a portion of the plurality of demographic groupings each correspond to at least one risk forecast model of the plurality of risk forecast models.

4. The system of claim 1, wherein the plurality of treatment data records comprises a plurality of medical insurance claims records.

5. The system of claim 1, wherein the operations further comprise segmenting the plurality of treatment data records according to a plurality of medical condition groupings, wherein the medical condition groupings each correspond to a respective preexisting medical condition of a plurality of preexisting medical conditions.

6. The system of claim 5, wherein the plurality of medical conditions comprises at least a portion of the plurality of preexisting medical conditions.

7. The system of claim 5, wherein the operations further comprise analyzing the plurality of treatment data records to identify, for one or more of the plurality of members, information identifying at least one of diagnosis or treatment of at least one medical condition of the plurality of medical conditions.

8. The system of claim 5, wherein the demographic data records comprise, for each member of a portion of the plurality of members, identification of one or more preexisting conditions of the plurality of preexisting medical conditions.

9. The system of claim 1, wherein prioritizing the at least one of the plurality of medical conditions or the plurality of members comprises identifying a top segment of the plurality of medical conditions according to greatest cost.

10. A system for deriving risk patterns corresponding to a set of medical conditions through analyzing historic treatment of a population of individuals, the system comprising:
a non-transitory computer readable storage region comprising one or more computer readable storage devices, the non-transitory computer readable storage region being configured to store a plurality of medical claims data records collected over at least a threshold period of time, wherein the plurality of medical claims data records relate to a plurality of members of a population; and
processing circuitry configured to perform operations comprising accessing the plurality of medical claims data records,
categorizing the plurality of medical claims data records according to one or more characteristics, wherein the one or more characteristics comprise at least one of a corresponding encounter type of a plurality of encounter types, a corresponding diagnostic type of a plurality of diagnostic types, a corresponding therapy type of a plurality of therapy types, or a corresponding medication type of a plurality of medication types,
identifying, within the plurality of medical claims data records, one or more codings indicative of each of a set of medical conditions,
identifying a plurality of risk patterns by analyzing, for each respective medical condition of the set of medical conditions, a portion of the plurality of medical claims data records corresponding to each member of the plurality of members of the population having codings indicative of the respective medical condition to recognize one or more risk patterns,
for each risk pattern of the plurality of risk patterns, calculating one or more impact values, each impact value corresponding to a resource allocation or a forecast outcome, and
arranging the plurality of risk patterns by impact according to at least one of the one or more impact values.

11. The system of claim 10, wherein the operations further comprise, after categorizing the plurality of medical claims data records, filtering the plurality of medical claims data records to remove one or more encounter types of the plurality of encounter types and/or one or more diagnostic types of the plurality of diagnostic types which is each representative of a respective pre-diagnosis event.

12. The system of claim 10, wherein the set of medical conditions comprise one or more of at least one disease, at least one chronic condition, at least one acute condition, or at least one disorder.

13. The system of claim 10, wherein the operations further comprise dimensionally reducing the plurality of risk patterns into a set of grouped risk patterns, wherein each group risk pattern represents a respective two or more risk patterns of the plurality of risk patterns having a common medical condition type.

14. The system of claim 13, wherein the common medical condition type is one of a type of disease, a type of disorder, a type of treatment, or a type of therapy.

15. The system of claim 13, wherein the common medical condition type comprises one or more of a cancer type, a cardiovascular type, a neurological type, an upper gastro-intestinal/esophageal type, a diabetes type, a musculoskeletal type, a hypertension type, a metabolic disorder type, a mental health disorder type, a lipid disorder type, a neurodivergent diagnoses type, or a pulmonary type.

16. The system of claim 13, wherein dimensionally reducing the plurality of risk patterns comprises aggregating a respective impact value of each risk pattern of the respective two or more risk patterns to calculate a group impact value.

17. The system of claim 10, wherein:
The non-transitory computer readable storage region is further configured to store a plurality of demographic data records comprising demographic information for each member of a plurality of members of a population, wherein each record of the plurality of demographic data records correlates to a corresponding portion of the plurality of medical claims data records; and
identifying the plurality of risk patterns comprises analyzing, for each respective medical condition of the set of medical conditions, the portion of the plurality of medical claims data records in view of demographic information derived from at least a portion of each of the demographic data records corresponding to the portion of the plurality of medical claims data records.

18. A system for deriving risk patterns corresponding to a set of medical conditions through analyzing historic treatment of a population of individuals, the system comprising:
a non-transitory computer readable storage region comprising one or more computer readable storage devices, the non-transitory computer readable storage region being configured to store
a plurality of treatment data records collected over at least a threshold period of time,
a plurality of demographic data records comprising demographic information for each member of a plurality of members of a population, wherein each record of the plurality of demographic data records correlates to a corresponding portion of the plurality of treatment data records, and
a plurality of risk forecast models trained, using historic claims data corresponding to a large population of individuals, to predict a likelihood of future expenditure on treatment data analysis, each risk forecast model configured to identify a respective at least one cost risk pattern corresponding to a respective at least one medical condition of a plurality of medical conditions; and
processing circuitry configured to perform operations comprising
accessing the plurality of treatment data records and the plurality of demographic data records,
segmenting the plurality of treatment data records into sets of segmented treatment data records according at least to a plurality of demographic groupings,
for each respective risk forecast model of the plurality of risk forecast models, applying one or more sets of the segmented claims data to the respective risk forecast model to calculate, for each individual of a plurality of individuals, a plurality of simulated cost forecasts, each cost forecast associated with at least one of a) a respective time period of a plurality of time periods, b) a respective magnitude of simulation outcome of a plurality of magnitudes of simulation outcomes, or c) a respective insurance overage plan of a plurality of insurance coverage plans,
dividing, according to the plurality of simulated cost forecasts, the plurality of individuals into a set of risk categories comprises at least a high risk, a medium risk, and a low risk, and
calculating, for each respective risk category of the set of risk categories, a respective number of individuals of the plurality of individuals categorized into the respective risk category, and at least one respective cost corresponding to the respective risk category.

19. The system of claim 18, wherein the plurality of magnitudes of simulation outcomes comprises a typical scenario and an extreme scenario.

20. The system of 18, wherein the at least one respective cost corresponding to the respective risk category comprises an average cost per each magnitude of simulation outcome of the plurality of magnitudes of simulation outcome.
